(19) United States
(12) Patent Application Publication
(10) Pub. No.: US 2002/0147306 A1
(43) Pub. Date: Oct. 10, 2002

(54) Peptides that modulate the interaction of B class ephrins and PDZ domains

(76) Inventors: Danny Lin, Scarborough (CA); Anthony Pawson, Toronto (CA); Gerald Gish, East York (CA)

Correspondence Address: ROPES & GRAY, ONE INTERNATIONAL PLACE, BOSTON, MA, 02110-2624, US

(21) Appl. No.: 09/862,179

(22) Filed: May 21, 2001

Related U.S. Application Data
(60) Provisional application No. 60/109,158, filed on Nov. 20, 1998.

(30) Foreign Application Priority Data
Nov. 19, 1999 (CA) PCT/CA99/01101

(51) Int. Cl. C07K 14/435

(52) U.S. Cl. 530/350; 530/324

(57) ABSTRACT

The invention relates to complexes comprising a B class ephrin and a PDZ domain containing protein; peptides that interfere with the interaction of a B class ephrin with a PDZ domain binding site, and a PDZ domain containing protein; and, uses of the peptides and complexes. Methods for modulating the interaction of a B class ephrin and a PDZ domain containing protein, and methods for evaluating compounds for their ability to modulate the interaction are also described.

### FIELD OF THE INVENTION

The invention relates to complexes comprising a B class ephrin with a PDZ domain binding site, and a PDZ domain containing protein; peptides that interfere with the interaction of a B class ephrin with a PDZ domain binding site, and a PDZ domain containing protein; and, uses of the peptides and complexes.

### BACKGROUND OF THE INVENTION

Among the large number of receptor tyrosine kinases (RTK) identified in metazoan organisms, the members of the Eph family are unusual in several respects. Although only one Eph RTK is known to be encoded by the Caenorhabditis elegans genome (the vab-1 gene product (2)), vertebrates typically possess up to 14 genes for Eph receptors, suggesting that these tyrosine kinases may be important in controlling complex cellular interactions (3,4). Consistent with this possibility, C. elegans VAB-1 regulates morphogenetic cell movements during ventral closure in the embryo (2), while vertebrate Eph receptors have been implicated in controlling axon guidance and fasciculation, in specifying topographic map formation within the central nervous system, in organizing the movements of neural crest cells during development, in directing fusion of epithelial sheets in closure of the palate, and in angiogenesis (5-15).

Early work on the expression patterns of EphB2 (formerly Nuk) suggested that this receptor is clustered at sites of cell-cell junctions in the developing mouse mid-brain, and raised the possibility that Eph receptors might mediate signals initiated by direct cell-cell interactions (5). Several lines of evidence support the notion that Eph receptors are normally activated by ligands that are physically associated with the surface of an adjacent cell. All known ligands for the Eph receptors (termed ephrins) are related in sequence, but can be divided into two groups based on their C-terminal motifs. The ephrin A class of ligands become modified by a C-terminal glycosylphosphatidyl inositol (GPI) moiety, through which the ligand is anchored to the surface of the ligand-expressing cell (7,9,16). In contrast, B-type ephrins possess a transmembrane element, and a highly conserved cytoplasmic tail comprised of 82-88 C-terminal residues (17-22). The Eph receptors can, in turn, be divided into A and B subgroups based on their sequence similarity and their propensity to bind soluble forms of either A or B type ephrins, respectively (4,23,24). However, although soluble ephrins bind tightly to the relevant receptors, consistent activation of Eph tyrosine kinase activity requires either that the ligands be artificially clustered into oligomers, or that receptor-expressing cells be co-cultured with cells expressing membrane-associated ephrins (18). These data suggest that the ability of ephrins to aggregate and thereby activate Eph receptors depends on their attachment to the cell surface, consistent with the view that Eph receptor signaling involves cell-cell interactions. During embryonic development in the mouse, Eph receptors and their ligands are expressed in dynamic but complementary patterns, indicating that Eph receptors are likely activated at boundaries where Eph and ephrin-expressing cells are directly juxtaposed to one another (23, 25).

Genetic analysis of Eph receptor function in C. elegans and the mouse has indicated that Eph receptors have both kinase-dependent and kinase-independent modes of signaling, and raised the possibility that B-type Eph receptors and ephrins might mediate bidirectional cell-to-cell signaling (2,6). Of interest, the binding of Eph receptors to transmembrane ephrin B1 or ephrin B2, as well as treatment of ephrin B-expressing cells with platelet-derived growth factor (PDGF), leads to the phosphorylation of the ephrins on tyrosine residues within their highly conserved cytoplasmic tails (26,27). Furthermore, expression of the cytoplasmic tail of a Xenopus ephrin B molecule leads to a striking loss of cell adhesion in Xenopus embryos, an effect that is suppressed by treatment with fibroblast growth factor (28).

### SUMMARY OF THE INVENTION

B class ephrins function as ligands for B class Eph receptor tyrosine kinases and possess an intrinsic signaling function. The sequence at the carboxy-terminus of B-type ephrins contains a PDZ binding site, providing a mechanism through which transmembrane ephrins interact with cytoplasmic proteins. A day 10.5 mouse embryonic expression library was screened with a biotinylated peptide corresponding to the C-terminus of ephrin B3. Three of the positive cDNAs encoded polypeptides with multiple PDZ domains, representing fragments of the glutamate receptor-interacting protein (GRIP) family, the protein syntenin and PHIP, a novel PDZ domain-containing protein related to Caenorhabditis elegans PAR-3. In addition, the binding specificities of PDZ domains previously predicted by an oriented library approach (1) identified the tyrosine phosphatase FAP-1 as a potential binding partner for B ephrins. In vitro studies demonstrated that the fifth PDZ domain of FAP-1 and full-length syntenin bound ephrin B1 via the C-terminal motif. Lastly, syntenin and ephrin B1 could be co-immunoprecipitated from transfected Cos-1 cells, indicating that PDZ domain binding of B ephrins occurs in cells. These results indicate that the C-terminal motif of B ephrins provides a binding site for specific PDZ domain-containing proteins, which potentially localize the transmembrane ligands for interactions with Eph receptors or participate in signaling within ephrin B-expressing cells.

Broadly stated the present invention relates to a complex comprising a B class ephrin and a PDZ domain containing protein. The invention is also directed to a peptide derived from the PDZ binding domain of a B class ephrin. The invention also contemplates antibodies specific for the complexes and peptides of the invention.

The present invention also provides a method of modulating the interaction of a B class ephrin and a PDZ domain containing protein comprising administering an effective amount of one or more of the following: (a) a complex comprising a B class ephrin and a PDZ domain containing protein; (b) a peptide derived from the PDZ binding domain of a B class ephrin; or, (c) enhancers or inhibitors of the interaction of a B class ephrin and a PDZ domain containing protein.

The invention still further provides a method for identifying a substance that binds to a complex comprising a B class ephrin B and a PDZ domain containing protein comprising: (a) reacting the complex with at least one substance which potentially can bind with the complex, under conditions which permit binding of the substance and complex; and (b) detecting binding, wherein detection of binding indicates the substance binds to the complex. Binding can be detected by assaying for substance-complex conjugates, or for activation of the B class ephrin B or PDZ domain containing protein. The invention also contemplates methods for identifying substances that bind to other intracellular proteins that interact with the complexes of the invention.

Still further the invention provides a method for evaluating a compound for its ability to modulate the interaction of a B class ephrin and a PDZ domain containing protein. For example, a substance that inhibits or enhances the interaction of the molecules in a complex of the invention, or a substance which binds to the molecules in a complex of the invention may be evaluated. In an embodiment, the method comprises providing a complex of the invention, with a substance which binds to the complex, and a test compound under conditions which permit the formation of conjugates between the substance and complex, and removing and/or detecting conjugates. In another embodiment, the method comprises providing a B class ephrin and a PDZ domain containing protein, and a test compound, under conditions which permit binding of the B class ephrin and PDZ domain containing protein; and (b) detecting binding, wherein the detection of increased or decreased binding relative to binding in the absence of the test compound indicates that the test compound modulates the interaction of a B class ephrin and a PDZ domain containing protein.

The present invention also contemplates a peptide of the formula I, herein after “PZD inhibiting peptide”, which interferes with the interaction of a B class ephrin and a PDZ domain containing protein

X-X¹-X²-K-V I

wherein X represents 0 to 70, preferably 0 to 50, more preferably 2 to 20 amino acids, and X¹ and X² each represent tyrosine or phosphotyrosine. The invention also relates to analogs of the peptides of the invention.

Further, the invention relates to a method of modulating the interaction of a B class ephrin and a PDZ domain containing protein comprising changing the terminal amino acid Val in a B class ephrin.

Another aspect of the invention provides a peptide or peptidomimetic of a PZD inhibiting peptide, e.g., wherein one or more backbone bonds is replaced or one or more sidechains of a naturally occurring amino acid are replaced with sterically and/or electronically similar functional groups.

In other embodiments, the invention provides a method for identifying inhibitors of the PZD-dependent interactions, comprising

(a) providing a reaction mixture including an ephrin or PDZ-binding domain thereof, and at least the PDZ domain of an intracellular protein which binds to the ephrin in a PDZ sequence-dependent manner;

(b) contacting the reaction mixture with one or more test compounds;

(c) identifying compounds which inhibit the interaction of PDZ domain and PDZ-binding domain.

In certain preferred embodiments, the reaction mixture is a whole cell. In other embodiments, the reaction mixture is a cell lysate or purified protein composition. The subject method can be carried out using libraries of test compounds. Such agents can be proteins, peptides, nucleic acids, carbohydrates, small organic molecules, and natural product extract libraries, such as isolated from animals, plants, fungus and/or microbes.

Still another aspect of the present invention provides a method of conducting a drug discovery business comprising:

(a) providing one or more assay systems for identifying agents by their ability to inhibit or potentiate the interaction of a PDZ domain and a PDZ-binding domain of an ephrin;

(b) conducting therapeutic profiling of agents identified in step (a), or further analogs thereof, for efficacy and toxicity in animals; and

(c) formulating a pharmaceutical preparation including one or more agents identified in step (b) as having an acceptable therapeutic profile.

In certain embodiments, the subject method can also include a step of establishing a distribution system for distributing the pharmaceutical preparation for sale, and may optionally include establishing a sales group for marketing the pharmaceutical preparation.

Yet another aspect of the invention provides a method of conducting a target discovery business comprising:

(a) providing one or more assay systems for identifying agents by their ability to inhibit or potentiate the interaction of a PDZ domain and a PDZ-binding domain of an ephrin;

(b) (optionally) conducting therapeutic profiling of agents identified in step (a) for efficacy and toxicity in animals; and

(c) licensing, to a third party, the rights for further drug development and/or sales for gents identified in step (a), or analogs thereof.

In one embodiment of the subject assay, the target intracellular protein is a glutamate receptor-interacting protein (GRIP). In other embodiments, the intracellular protein is a syntenin, e.g., a PDZ protein that binds the cytoplasmic C-terminal FYA motif of the syndecans. In still other embodiments, the intracellular protein is an ephrin interacting protein or “PHIP”. PHIP proteins are characterized by the presence of two or more adjacent PDZ domains followed by a 50-amino acid carboxyl-terminal stretch.

In certain preferred embodiments, the subject drug screening methods can be used to identify inhibitors having molecular weights less than 5000 amu, more preferably less than 2500 amu, and most preferably less than 1000 amu, e.g, to identify small organic molecule inhibitors.

In certain preferred embodiments, the subject drug screening methods can be used to identify inhibitors having Ks's for ephrin-PDZ interactions of less than 25 μM, and more preferably less than 1 μM, 100 nM, 10 nM or even 1 nM.

The complexes, peptides and antibodies of the invention, and substances and compounds identified using the methods of the invention may be used to modulate the interaction of a B class ephrin and a PDZ domain containing protein, and they may be used to modulate cellular processes of cells associated with B class ephrins and/or PDZ domain containing proteins (such as proliferation, growth, and/or differentiation, in particular axonogenesis, nerve cell interactions and regeneration) in which the compounds or substances are introduced.

Accordingly, the complexes, antibodies, peptides, substances and compounds may be formulated into compositions for administration to individuals suffering from disorders associated with a B class ephrin such as disorders of the central nervous system (e.g. neurodegenerative diseases and cases of nerve injury). Therefore, the present invention also relates to a composition comprising one or more of a complex, peptide, or antibody of the invention, or a substance or compound identified using the methods of the invention, and a pharmaceutically acceptable carrier, excipient or diluent. A method for modulating proliferation, growth, and/or differentiation of cells associated with B class ephrins and/or PDZ domain containing proteins is also provided comprising introducing into the cells a complex, peptide or antibody of the invention, a compound or substance identified using the methods of the invention or a composition containing same. Methods for treating proliferative and/or differentiative disorders associated with B class ephrins and/or PDZ domain containing proteins using the compositions of the invention are also provided.

Diseases in which inhibition of PDZ-mediated ephrin interactions can provide therapeutic benefit include: arthritis (including osteoarthritis and rheumatoid arthritis), inflammatory bowel disease, Crohn's disease, emphysema, acute respiratory distress syndrome, asthma chronic obstructive pulmonary disease, Alzheimer's disease, organ transplant toxicity, cachexia, allergic reactions, allergic contact hypersensitivity, cancer, tissue ulceration, restenosis, periodontal disease, epidermolysis bullosa, osteoporosis, loosening of artificial joint implants, atherosclerosis (including atherosclerotic plaque rupture), aortic aneurysm (including abdominal aortic aneurysm and brain aortic aneurysm), congestive heart failure, myocardial infarction, stroke, cerebral ischemia, head trauma, spinal cord injury, neuro-degenerative disorders (acute and chronic), autoimmune disorders, Huntington's disease, Parkinson's disease, migraine, depression, peripheral neuropathy, pain, cerebral amyloid angiopathy, nootropic or cognition enhancement, amyotrophic lateral sclerosis, multiple sclerosis, ocular angiogenesis, corneal injury, macular degeneration, abnormal wound healing, bums, diabetes, tumor invasion, tumor growth, tumor metastasis, corneal scarring, scleritis, AIDS, sepsis, and septic shock.

Other objects, features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will now be described in relation to the drawings in which:

FIG. 1. Amino acid sequence of the cytoplasmic domains of the human B ephrins. Conserved residues among the three B ephrins are highlighted. Asterisks mark conserved tyrosines that are potential sites of phosphorylation. The potential PDZ domain binding site is underlined.

FIGS. 2A-D. Identification of PDZ domain-containing candidates for ephrin B binding. FIG. 2A, The preferred binding sequence of FAP-1 PDZ5 is shown below a schematic representation of the entire FAP-1 protein tyrosine phosphatase. FAP-1 PDZ5 domain specificity was deduced from an oriented peptide library technique (1). Residues within the optimal binding sequence that match the C-terminal sequence of B ephrins are indicated in bold. The organization of the PDZ domains of FAP-1 shown in this figure follows the numbering described by Sato et al. (33). FIG. 2B, Diagrammatic representations of the PDZ domain-containing proteins identified through an expression screen with a biotinylated peptide probe of eprhin B3 C-terminal sequence. The brackets mark the portions of the protein encoded by the cDNAs isolated from the screen. PDZ domains are represented by grey boxes. FIG. 2C, Amino acid sequence alignment of FAP-1 PDZ5 and of the PDZ domains isolated in the expression screen. The numbering of the PDZ domains is as shown in FIG. 2B. Conserved residues are highlighted. The alignment was performed with the ClustalW program (55). FIG. 2D, Amino acid sequence alignment of PHIP and PAR-3. Conserved residues are highlighted and the PDZ domains are underlined. The alignment was performed with the Genestream Align program.

FIGS. 3A-C. FAP-1 PDZ5 and syntenin bind specifically to ephrin B1 in GST-mixes. Cos-1 cells were transiently transfected with either wild-type ephrin B1 (W. T.) or the ephrin B1 Val deletion (Val Δ) or were untransfected. Cell lysates were incubated with the GST fusion proteins as indicated and analyzed by immunoblotting with anti-ephrin B1 antibody. Immunoprecipitated ephrin B1 or ephrin B1 Val Δ were included as a positive control. FIG. 3A and FIG. 3B, GST-mixes with fusion proteins of FAP-1. C and D, GST-mixes with fusion proteins of syntenin.

FIGS. 4A and 4B. FAP-1 PDZ5 and syntenin binding to ephrin B1 can be blocked by addition of peptides corresponding to the C-terminal sequence of B ephrins. Peptides of the indicated sequence were included at a concentration of 100 μM in incubations of GST fusion proteins with lysates of Cos-1 cells transfected with ephrin B1. Associated proteins were separated on a 10% polyacrylamide/SDS gel and analyzed by immunoblotting with antibodies against ephrin B1. FIG. 4A, Competition of FAP-1 PDZ5 binding to ephrin B1 using the indicated peptides. A peptide of sequence DHQpYpYND was added at a concentration of 100 μM as a negative control. Immunoprecipitation of ephrin B1 was included as a positive control. FIG. 4B, Peptide competition of the binding of full-length syntenin to ephrin B1.

FIGS. 5A and 5B. Fluorescence polarization analysis of GST-FAP-1 PDZ3, GST-FAP-1 PDZ5 and GST-syntenin binding to Fluorescein-labelled peptides corresponding to the C-terminus of ephrin B1. FIG. 5 A, Solutions containing the indicated final concentration of GST-FAP-1 PDZ3 ( [symbol] ) or GST-FAP-1 PDZ5 ( [symbol] ) fusion protein in mixtures containing 25 nM fluorescein-labelled NIYYKV peptide probe, 20 mM phosphate pH 7.0, 100 mM NaCl, and 2 mM dithiothreitol (DTT) were monitored for fluorescence polarization at 22° C. The GST-FAP-1 PDZ5 fusion protein was also measured for binding to the phosphorylated peptides, NIpYYKV ( ), NiYpYKV ( ) and NIpYpYKV ( [symbol] ). The fluorescence polarization values obtained for the peptide in absence of added GST-fusion protein has been subtracted from the polarization values displayed. FIG. 5B, A Binding of a GST fusion of full-length syntenin to the NIYYKV ( [symbol] ), NIpYYKV ( ), and NIpYpYKV ( [symbol] ) peptides as measured by fluorescence polarization.

FIG. 6. Co-immunoprecipitation of syntenin-FLAG with ephrin B1. Cos-1 cells were co-transfected with either ephrin B1 and syntenin-FLAG or with the ephrin B1 Val deletion and syntenin-FLAG as indicated. Cell lysates were immunoprecipitated with antibodies against ephrin B1 or IL-3 receptor a or were treated with protein A sepharose only. Immunocomplexes were subjected to SDS-PAGE (10%) and blotted with anti-FLAG antibodies.

FIG. 7. Fluorescence polarization analysis of GST-PHIP PDZ3 binding to Fluorescein-labelled peptides corresponding to the C-terminus of ephrin B1. Solutions containing the indicated final concentration of GST-PHIP PDZ3 fusion protein in mixtures containing 25 nM fluorescein-labelled peptide probe, 20 mM phosphate pH 7.0, 100 mM NaCl, and 2 mM DTT were monitored for fluorescence polarization at 22° C. The GST-PHIP PDZ3 fusion protein was measured for binding to the phosphorylated peptides, NIpYYKV ( ), NiYpYKV ( ) and NIpYpYKV ( [symbol] ) and the unphosphorylated NIYYKV peptide ( [symbol] ). The fluorescence polarization values obtained for the peptide in absence of added GST-fusion protein has been subtracted from the polarization values displayed.

FIG. 8 PHIP PDZ3 binds specifically to V-Src phosphorylated ephrin B1 in GST-mixes. COS-1 cells were transiently co-transfected with V-Src and either wild-type ephrin B1 or the ephrin B1 Val deletion (VA) or were transfected with either wild-type ephrin B1 or ephrin B1 Val deletion alone. Cell lysates were incubated with the GST fusion proteins as indicated and analyzed by immunoblotting with anti-phosphotyrosine antibody. Immunoprecipitated ephrin B1 was included as a positive control.

### DETAILED DESCRIPTION OF THE INVENTION

Definitions

Unless otherwise indicated, all terms used herein have the same meaning as they would to one skilled in the art of the present invention. Practitioners are particularly directed to Current Protocols in Molecular Biology (Ansubel) for definitions and terms of the art.

Abbreviations for amino acid residues are the standard 3-letter and/or 1-letter codes used in the art to refer to one of the 20 common L-amino acids. Likewise abbreviations for nucleic acids are the standard codes used in the art.

“Antibody” refers to intact monoclonal or polyclonal molecules, and immunologically active fragments (e.g. a Fab or (Fab)₂ fragment), an antibody heavy chain, and antibody light chain, a genetically engineered single chain Fᵥ molecule (Ladner et al, U.S. Pat. No. 4,946,778), or a chimeric antibody, for example, an antibody which contains the binding specificity of a murine antibody, but in which the remaining portions are of human origin. Antibodies including monoclonal and polyclonal antibodies, fragments and chimeras, may be prepared using methods known to those skilled in the art. Antibodies that bind a complex, or peptide of the invention can be prepared using intact peptides or fragments containing an immunizing antigen of interest. The polypeptide or oligopeptide used to immunize an animal may be obtained from the translation of RNA or synthesized chemically and can be conjugated to a carrier protein, if desired. Suitable carriers that may be chemically coupled to peptides include bovine serum albumin and thyroglobulin, keyhole limpet hemocyanin. The coupled peptide may then be used to immunize the animal (e.g., a mouse, a rat, or a rabbit).

“B class ephrin” refers to a family of proteins that bind Eph receptors and possess a transmembrane element, and a highly conserved cytoplasmic tail comprised of 82-88 C-terminal residues (17-22). Examples of B class ephrins include ephrin B1 (also known as LERK-2, Elk-L, EFL-3, Cek-L, and STRA1), ephrin B2 (also known as Htk-L, ELF-2, LERK-5, and NLERK-1), and ephrin B3 (also known as NLERK-2, Elk-L3, EFL-6, ELF-3, and LERK-8). The family also includes proteins with substantial sequence identity (i.e. homologs) and portions of the proteins (e.g. see SEQ. ID. NO. 15, 16, or 17). The B class ephrins used in the complexes and methods of the invention contain a binding domain that binds a PDZ domain containing protein. The binding domain contains the consensus sequence YYKV.

The term “isolated”, as used herein, refers to nucleic or amino acid sequences that are removed from their natural environment, isolated or separated, and are at least 60% free, preferably 75% free, and most preferably 90% free from other components with which they are naturally associated.

The term “modulate”, as used herein, refers to a change or an alteration in the biological activity of a protein. Modulation may be an increase or a decrease in protein activity, a change in binding characteristics, or any other change in the biological, functional, or immunological properties of a protein.

The term “agonist” as used herein, refers to a molecule which when bound to a complex of the invention or a molecule in the complex, increases the amount of, or prolongs the duration of, the activity of a B class ephrin or PDZ domain containing protein, or increases complex formation. Agonists may include proteins, nucleic acids, carbohydrates, or any other molecules that bind to a complex or molecule of the complex. Agonists also include a peptide or peptide fragment derived from the PDZ binding domain of a B class ephrin but will not include the full length sequence of the wild-type molecule. Peptide mimetics, synthetic molecules with physical structures designed to mimic structural features of particular peptides, may serve as agonists. The stimulation may be direct, or indirect, or by a competitive or non-competitive mechanism.

The term “antagonist”, as used herein, refers to a molecule which, when bound to a complex of the invention or a molecule in the complex, decreases the amount of or duration of the activity of a B class ephrin or PDZ domain containing protein, or decreases complex formation. Antagonists may include proteins, nucleic acids, carbohydrates, or any other molecules that bind to a B class ephrin or PDZ domain containing protein. Antagonists also include a peptide or peptide fragment derived from the PDZ binding domain of a B class ephrin but will not include the full length sequence of the wild-type molecule. Peptide mimetics, synthetic molecules with physical structures designed to mimic structural features of particular peptides, may serve as antagonists. The inhibition may be direct, or indirect, or by a competitive or non-competitive mechanism.

“PDZ domain containing protein” refers to proteins or peptides, or parts thereof which comprise or consist of a characteristic structural motif known as the PDZ domain. (See the Structural Classification of Proteins (SCOP) database for the characteristics of the domain.) Examples of the proteins include GRIP, syntenin, and FAP-1, and homologs or portions thereof. Other proteins containing PDZ domains may be selected using public databases such as GENPEPT and ENTREZ. The present inventors isolated a novel PDZ domain containing protein designated “PHIP” as more particularly described herein. Examples of PDZ domain containing proteins include GRIP, GRIP PDZ6 and PDZ 7 of SEQ.ID.NO.22 and 23, FAP-1 PDZ5 of SEQ. ID. NO.21, amino acids residues 1 to 299 of syntenin, syntenin PDZ1 and PDZ2 of SEQ. ID. NO.26 and 27, PHIP PDZ2 of SEQ. ID. NO.24, and PHIP PDZ3 of SEQ. ID. NO. 25.

A “binding domain” is that portion of the molecule in a complex of the invention which interacts directly or indirectly with another molecule in a complex of the invention. The binding domain may be a sequential portion of the molecule i.e. a contiguous sequence of amino acids, or it may be conformational i.e. a combination of non-contiguous sequences of amino acids which when the molecule is in its native state forms a structure that interacts with another molecule in a complex of the invention.

By being “derived from” a binding domain is meant any molecular entity which is identical or substantially equivalent to the native binding domain of a molecule in a complex of the invention. A peptide derived from a specific binding domain may encompass the amino acid sequence of a naturally occurring binding site, any portion of that binding site, or other molecular entity that functions to bind to an associated molecule. A peptide derived from such a binding domain will interact directly or indirectly with an associated molecule in such a way as to mimic the native binding domain. Such peptides may include competitive inhibitors, peptide mimetics, and the like.

The term “interacting” refers to a stable association between two molecules due to, for example, electrostatic, hydrophobic, ionic and/or hydrogen-bond interactions under physiological conditions. Certain interacting molecules interact only after one or more of them has been stimulated. For example, a PDZ domain containing protein may only bind to a substrate if the substrate is phosphorylated (eg. phosphorylated).

“Peptide mimetics” are structures which serve as substitutes for peptides in interactions between molecules (See Morgan et al (1989), Ann. Reports Med. Chem. 24:243-252 for a review ). Peptide mimetics include synthetic structures which may or may not contain amino acids and/or peptide bonds but retain the structural and functional features of a peptide, or agonist or antagonist of the invention. Peptide mimetics also include peptoids, oligopeptoids (Simon et al (1972) Proc. Natl. Acad, Sci USA 89:9367); and peptide libraries containing peptides of a designed length representing all possible sequences of amino acids corresponding to a peptide, or agonist or antagonist of the invention.

The following terms are used to describe the sequence relationships between two or more nucleic acid molecules or proteins: “reference sequence”, and “substantial sequence identity”. A “reference sequence” is a defined sequence used as a basis for a sequence comparison; a reference sequence may be a subset of a larger sequence, for example, a segment of a full-length cDNA or gene sequence given in a sequence listing or may comprise a complete cDNA or gene sequence. Optimal alignment of sequences for aligning a comparison window may be conducted by the local homology algorithm of Smith and Waterman (1981) Adv. Appl. Math. 2:482, by the homology alignment algorithm of Needleman and Wunsch (1970) J. Mol. Biol. 48:443, by the search for similarity method of Pearson and Lipman (1988) Proc. Natl. Acad. Sci. (USA) 85:2444, or by computerized implementations of these algorithms (GAP, BESTFIT, FASTA, and TFASTA in the Wisconsin Genetics Software Package Release 7.0, Genetics Computer Group, 575 Science Dr., Madison, Wis.; ClustalW program (55); and the Genestream Align Program). As applied to polypeptides, the term “substantial sequence identity” means that two peptide sequences, when optimally aligned, such as by the programs GAP or BESTFIT using default gap share at least 90 percent sequence identity, preferably at least 95 percent sequence identity, more preferably at least 99 percent sequence identity or more. Preferably, residue positions which are not identical differ by conservative amino acid substitutions. For example, the substitution of amino acids having similar chemical properties such as charge or polarity are not likely to effect the properties of a protein. Examples include glutamine for asparagine or glutamic acid for aspartic acid.

Complexes of the Invention

The complexes of the invention comprise a B class ephrin protein and a PDZ domain containing protein. It will be appreciated that the complexes may comprise only the binding domains of the interacting molecules and such other flanking sequences as are necessary to maintain the activity of the complexes.

In an embodiment of the invention, the PDZ domain containing protein in the complex is GRIP, GRIP PDZ6 and PDZ 7 of SEQ.ID.NO. 22 and 23, FAP-1 PDZ of SEQ. ID. NO. 21, amino acids residues 1 to 299 of syntenin, syntenin PDZ1 and PDZ2 of SEQ. ID. NO. 26 and 27, PHIP PDZ2 of SEQ. ID. NO. 24, and PHIP PDZ3 of SEQ. ID. NO. 25. Examples of complexes of the invention include ephrin B3/GRIP; ephrin B3/GRIP PDZ6 and PDZ 7 of SEQ.ID.NO. 22 and 23; ephrin B1/FAP-1 PDZ of SEQ. ID. NO. 21; ephrin B1 or B3/amino acids residues 1 to 299 of syntenin; ephrin B1 or B3/ syntenin PDZ1 and PDZ2 of SEQ. ID. NO.26 and 27; ephrin B1 or B3/PHIP PDZ2 of SEQ. ID. NO. 24, and ephrin B1 or B3/PHIP PDZ3 of SEQ. ID. NO. 25. The complexes may comprise a portion of the B class ephrin, or a peptide of the invention. For example, the complex may comprise YYKV (SEQ ID. NO. 5), GPPQSPPNIpYYKV (SEQ ID. NO. 6), NIpYpYKV (SEQ ID. NO. 7), NIpYYKV (SEQ ID. NO. 8), NIYpYKV (SEQ ID. NO. 9), NIYYKV (SEQ ID. NO. 10), GNIYYKV (SEQ ID. NO. 28), GNIpYpYKV (SEQ ID. NO. 29), GNIpYYKV (SEQ ID. NO. 30 ), and GNIYpYKV (SEQ ID. NO. 31). Examples of such complexes include FAP-1 PDZ/NIYYKV, syntenin/NIYYKV, syntenin PDZ1 and PDZ2/NIYYKV, PHIP PDZ3/GNIYYKV, and PHIP PDZ2/GNIYYKV.

As illustrated herein the B class ephrin or portion thereof, or peptide of the invention, in a complex of the invention may be phosphorylated. Therefore, a complex of the invention comprising a PDZ domain containing protein as one component may comprise a phosphorylated B class ephrin or a portion thereof, or a phosphorylated peptide of the invention as another component. For example, the complex may comprise FAP-1 PDZ/NIpYYKV, FAP-1 PDZ/NIpYpYKV, syntenin/NIYYKV, syntenin/NIpYYKV, syntenin PDZ1 and PDZ2/NIYYKV, syntenin PDZ1 and PDZ2/ NIpYYKV, PHIP PDZ3/GNIpYpYKV, and PHIP PDZ3/GNIpYYKV.

The invention also contemplates antibodies specific for complexes of the invention. The antibodies may be intact monoclonal or polyclonal antibodies, and immunologically active fragments (e.g. a Fab or (Fab)₂ fragment), an antibody heavy chain, and antibody light chain, a genetically engineered single chain Fᵥ molecule (Ladner et al, U.S. Pat. No. 4,946,778), or a chimeric antibody, for example, an antibody which contains the binding specificity of a murine antibody, but in which the remaining portions are of human origin. Antibodies including monoclonal and polyclonal antibodies, fragments and chimeras, may be prepared using methods known to those skilled in the art.

Antibodies specific for the complexes of the invention may be used to detect the complexes in tissues and to determine their tissue distribution. In vitro and in situ detection methods using the antibodies of the invention may be used to assist in the prognostic and/or diagnostic evaluation of proliferative and/or differentiative disorders associated with a B class ephrin e.g. disorders of the nervous system. Some genetic diseases may include mutations at the binding domain regions of the interacting molecules in the complexes of the invention. Therefore, if a complex of the invention is implicated in a genetic disorder, it may be possible to use PCR to amplify DNA from the binding domains to quickly check if a mutation is contained within one of the domains. Primers can be made corresponding to the flanking regions of the domains and standard sequencing methods can be employed to determine whether a mutation is present. This method does not require prior chromosome mapping of the affected gene and can save time by obviating sequencing the entire gene encoding a defective protein.

PHIP Protein

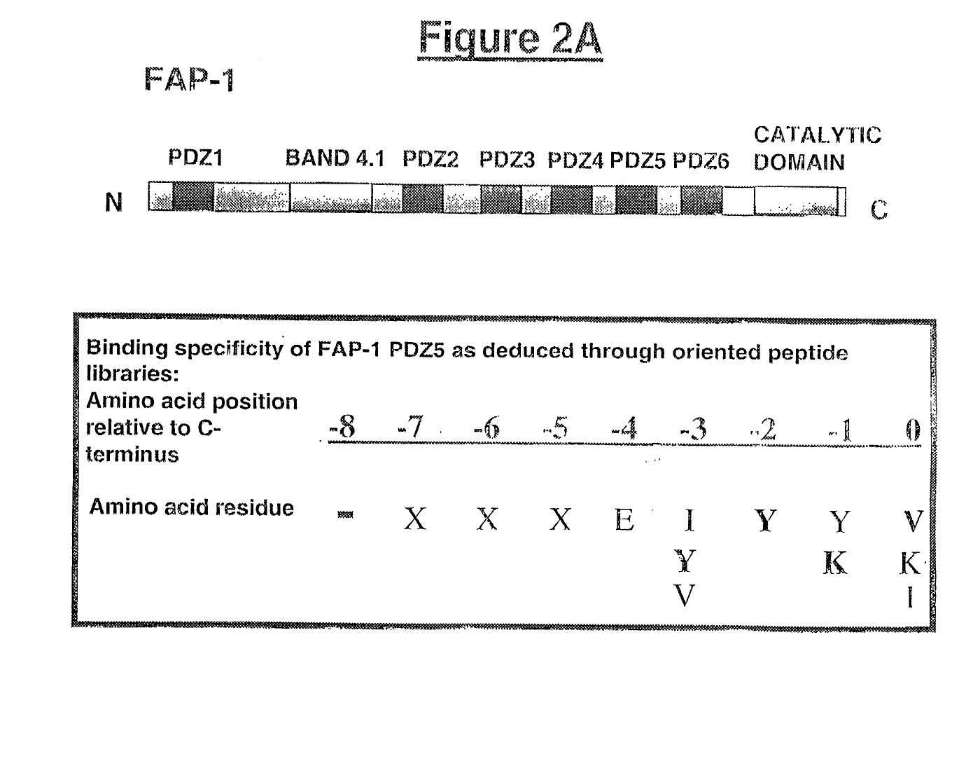

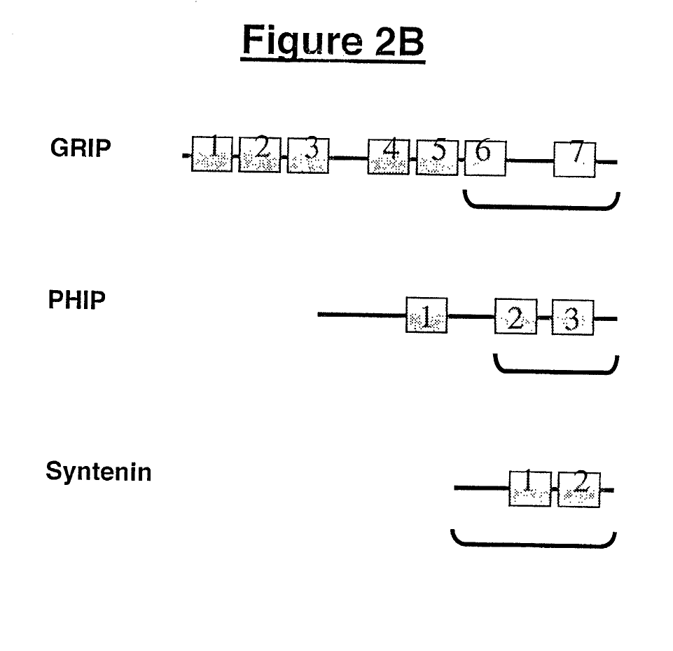

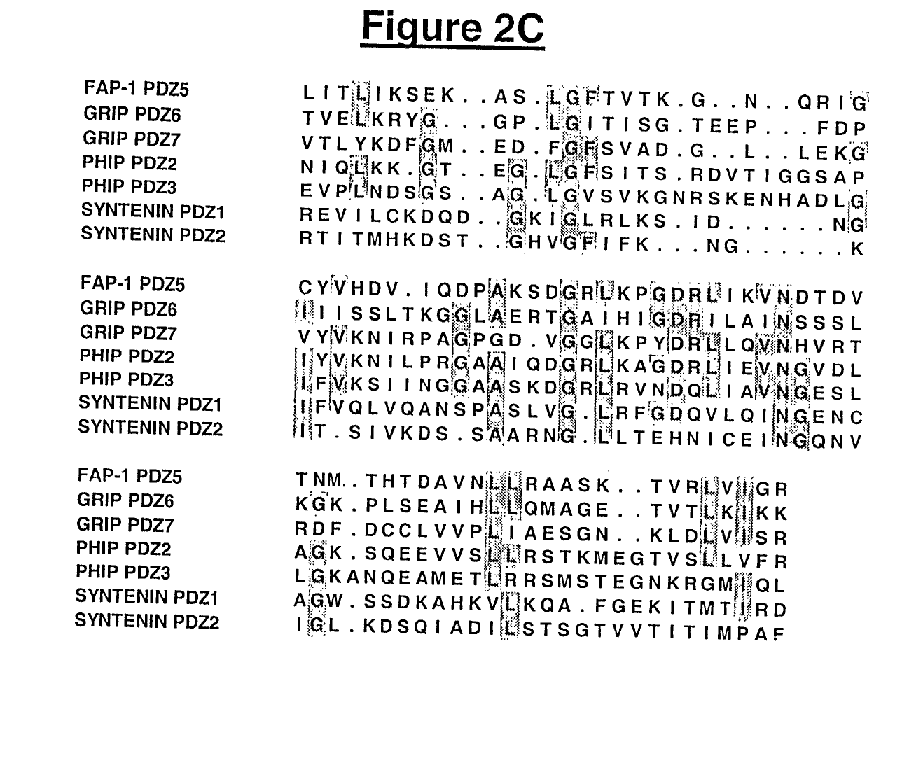

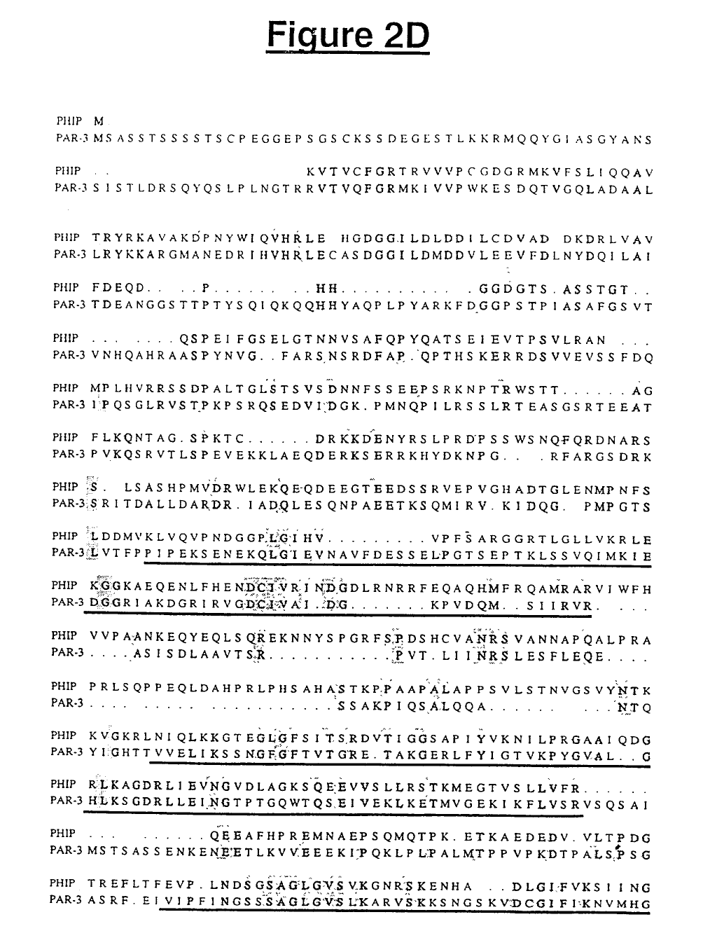

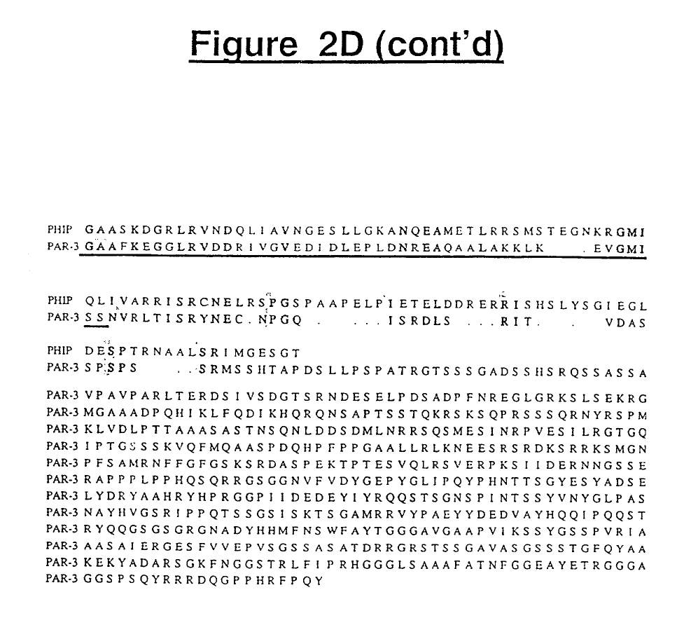

Broadly stated the present invention contemplates an isolated protein comprising the amino acid sequence shown in FIG. 2D and in SEQ. ID. NO.1. The invention contemplates a truncation (i.e. portion) of a protein of the invention, an analog, an allelic or species variation thereof, or a protein having substantial sequence identity with a protein of the invention (i.e. homolog), or a truncation thereof. (Truncations, analogs, allelic or species variations, and homologs are collectively referred to herein as “PHIP Related Proteins”).

Truncated proteins may comprise peptides of between 3 and 70 amino acid residues, ranging in size from a tripeptide to a 70 mer polypeptide, preferably 12 to 20 amino acids. In one aspect of the invention, fragments of PHIP protein are provided having an amino acid sequence of at least five consecutive amino acids in FIG. 2D and in SEQ. ID. NO. 1, where no amino acid sequence of five or more, six or more, seven or more, or eight or more, consecutive amino acids present in the fragment is present in a protein other than a PHIP Protein. In an embodiment of the invention the fragment is a stretch of amino acid residues of at least 12 to 20 contiguous amino acids from a particular sequence such as a sequence underlined in FIG. 2D. The fragments may be immunogenic and preferably are not immunoreactive with antibodies that are immunoreactive to proteins other than a PHIP protein.

In an aspect of the invention, isolated nucleic acids are provided comprising sequences encoding PHIP protein or PHIP Related Proteins.

The nucleic acids of the invention may be inserted into an appropriate vector, and the vector may contain the necessary elements for the transcription and translation of an inserted coding sequence. Accordingly, vectors may be constructed which comprise a nucleic acid of the invention, and where appropriate one or more transcription and translation elements linked to the nucleic acid molecule.

A vector of the invention can be used to prepare transformed host cells expressing a PHIP protein or a PHIP Related Protein. Therefore, the invention further provides host cells containing a vector of the invention.

The invention also contemplates transgenic non-human mammals whose germ cells and somatic cells contain a recombinant molecule comprising a nucleic acid molecule of the invention in particular one that encodes an analog of a PHIP protein, or a truncation of a PHIP protein.

A PHIP protein or PHIP Related Protein may be obtained as an isolate from natural cell sources, but they are preferably produced by recombinant procedures. In one aspect the invention provides a method for preparing a PHIP protein or a PHIP Related Protein utilizing an isolated nucleic acid molecule of the invention. In an embodiment a method for preparing a PHIP protein or a PHIP Related Protein is provided comprising:

(a) transferring a vector of the invention having a nucleotide sequence encoding a PHIP protein or PHIP Related Protein, into a host cell;

(b) selecting transformed host cells from untransformed host cells;

(c) culturing a selected transformed host cell under conditions which allow expression of the PHIP protein or PHIP Related Protein and

(d) isolating the PHIP protein or PHIP Related Protein.

The invention further broadly contemplates a recombinant PHIP protein or PHiP Related Protein obtained using a method of the invention.

A PHIP protein or PHIP Related Protein of the invention may be conjugated with other molecules, such as proteins, to prepare fusion proteins or chimeric proteins. This may be accomplished, for example, by the synthesis of N-terminal or C-terminal fusion proteins.

The invention further contemplates antibodies having specificity against an epitope of a PHIP protein or PHIP Related Protein of the invention. Antibodies may be labeled with a detectable substance and used to detect proteins of the invention in tissues and cells.

The invention also permits the construction of nucleotide probes which are unique to the nucleic acid molecules of the invention and accordingly to proteins of the invention. Therefore, the invention also relates to a probe comprising a nucleic acid sequence encoding a protein of the invention, or a part thereof. The probe may be labeled, for example, with a detectable substance and it may be used to select from a mixture of nucleotide sequences a nucleic acid molecule of the invention including nucleic acid molecules coding for a protein which displays one or more of the properties of a protein of the invention.

The invention still further provides a method for identifying a substance which binds to a protein of the invention comprising reacting the protein with at least one substance which potentially can bind with the protein, under conditions which permit the binding of the substance and protein; and detecting binding, wherein the detection of binding indicates that the substance binds to the protein. Binding can be detected by assaying for protein-substance complexes, or for activation of the protein. The invention also contemplates methods for identifying substances that bind to other intracellular proteins that interact with a PHIP protein or a PHIP Related Protein. Methods can also be utilized which identify compounds which bind to gene regulatory sequences (e.g. promoter sequences).

Still further the invention provides a method for evaluating a compound for its ability to modulate the biological activity of a PHIP protein or a PHIP Related Protein of the invention. For example, the compound may be a substance that binds to the proteins or a substance that inhibits or enhances the interaction of the protein and a substance that binds to the protein (e.g. a B class ephrin). In an embodiment, the method comprises providing a PHIP protein or a PHIP Related Protein, a substance which binds to the protein, and a test compound under conditions which permit binding of the substance and protein, and detecting binding, wherein the detection of increased or decreased binding relative to binding detected in the absence of the test compound indicates that the test compound modulates the activity of a PHIP protein or a PHIP Related Protein. Binding may be detected by assaying for substance-protein complexes, free substance, and/or free protein, or activation of the protein.

Activation of PHIP or a PHIP Related Protein may be assayed by measuring phosphorylation of the protein, or binding of the protein to cellular proteins, or by assaying for a biological affect on the cell, such as inhibition or stimulation of proliferation, differentiation, or migration.

Compounds which modulate the biological activity of a protein of the invention may also be identified using the methods of the invention by comparing the pattern and level of expression of a PHIP protein or a PHIP Related Protein of the invention in tissues and cells, in the presence, and in the absence of the compounds.

The substances and compounds identified using the methods of the invention may be used to modulate the biological activity of a PHIP protein or a PHIP Related Protein of the invention, and they may be used in the treatment of conditions requiring modulation of the proteins or other molecules that bind to a PHIP protein or a PHIP Related Protein (e.g. a B class ephrin).

Peptides

The invention provides peptide molecules that bind to and inhibit the interactions of the molecules in the complexes of the invention. The molecules are derived from the binding domain of a B class ephrin that binds to a PDZ domain containing protein. For example, peptides of the invention include the amino acids YYKV of ephrin B1, B2 or B3 that bind to a PDZ domain containing protein. Other proteins containing these binding domain sequences may be identified with a protein homology search, for example by searching available databases such as GenBank or SwissProt and various search algorithms and/or programs may be used including FASTA, BLAST (available as a part of the GCG sequence analysis package, University of Wisconsin, Madison, Wis.), or ENTREZ (National Center for Biotechnology Information, National Library of Medicine, National Institutes of Health, Bethesda, Md.).

In accordance with an embodiment of the invention, specific peptides are contemplated that mediate the binding of a B class ephrin and a PDZ domain containing protein. In particular, a PDZ inhibitory peptide of the formula I is provided which interferes with the interaction of a B class ephrin and a PDZ domain containing protein:

X-X¹-X²-K-V I

wherein X represents 0 to 70, preferably 0 to 50 amino acids, more preferably 2 to 20 amino acids, and Xl and X² each represent tyrosine or phosphotyrosine. In specific embodiments, X¹ is tyrosine and X² is phosphotyrosine, X¹ is phosphotyrosine and X² is tyrosine, or X¹ and X² are phosphotyrosine.

In an embodiment of the present invention a peptide of the formula I is provided where X represents NI, GNI, CPHYEKVSGDYGHPVYIVQ(E,D)(M,G)PPQSP(A,P)A (SEQ.ID. NO. 2), GDYGHPVYIVQ(E,D)(M,G)PPQSP(A,P)A (SEQ.ID. NO. 3), PPQSP(A,P)A (SEQ.ID. NO. 4), GPPQSPPNI (SEQ.ID. NO. 32).

Preferred peptides of the invention include the following: YYKV (SEQ ID. NO. 5), GPPQSPPNIpYYKV (SEQ ID. NO. 6), NIpYpYKV (SEQ ID. NO. 7), NIpYYKV (SEQ ID. NO. 8), NIYpYKV (SEQ ID. NO. 9), NTYYKV (SEQ ID. NO. 10), GNIYYKV (SEQ ID. NO. 28), GNIpYpYKV (SEQ ID. NO. 29), GNIpYYKV (SEQ ID. NO. 30 ), and GNIYpYKV (SEQ ID. NO. 31).

All of the peptides of the invention, as well as molecules substantially homologous, complementary or otherwise functionally or structurally equivalent to these peptides may be used for purposes of the present invention. In addition to full-length peptides of the invention, truncations of the peptides are contemplated in the present invention. Truncated peptides may comprise peptides of about 7 to 10 amino acid residues

The truncated peptides may have an amino group (—NH2), a hydrophobic group (for example, carbobenzoxyl, dansyl, or T-butyloxycarbonyl), an acetyl group, a 9-fluorenylmethoxy-carbonyl (PMOC) group, or a macromolecule including but not limited to lipid-fatty acid conjugates, polyethylene glycol, or carbohydrates at the amino terminal end. The truncated peptides may have a carboxyl group, an amido group, a T-butyloxycarbonyl group, or a macromolecule including but not limited to lipid-fatty acid conjugates, polyethylene glycol, or carbohydrates at the carboxy terminal end.

The peptides of the invention may also include analogs of a peptide of the invention and/or truncations of the peptide, which may include, but are not limited to the peptide of the invention containing one or more amino acid insertions, additions, or deletions, or both. Analogs of the peptide of the invention exhibit the activity characteristic of the peptide e.g. interference with the interaction of a B class ephrin and a PDZ domain containing protein, and may further possess additional advantageous features such as increased bioavailability, stability, or reduced host immune recognition. One or more amino acid insertions may be introduced into a peptide of the invention. Amino acid insertions may consist of a single amino acid residue or sequential amino acids.

One or more amino acids, preferably one to five amino acids, may be added to the right or left termini of a peptide of the invention. Deletions may consist of the removal of one or more amino acids, or discrete portions from the peptide sequence. The deleted amino acids may or may not be contiguous. The lower limit length of the resulting analog with a deletion mutation is about 7 amino acids.

It is anticipated that if amino acids are inserted or deleted in sequences outside an NIX¹X¹KV sequence that the resulting analog of the peptide will exhibit the activity of a peptide of the invention.

The invention also includes a peptide conjugated with a selected protein, or a selectable marker (see below) to produce fusion proteins.

The peptides of the invention may be prepared using recombinant DNA methods. Accordingly, nucleic acid molecules which encode a peptide of the invention may be incorporated in a known manner into an appropriate expression vector which ensures good expression of the peptide. Possible expression vectors include but are not limited to cosmids, plasmids, or modified viruses so long as the vector is compatible with the host cell used. The expression vectors contain a nucleic acid molecule encoding a peptide of the invention and the necessary regulatory sequences for the transcription and translation of the inserted protein-sequence. Suitable regulatory sequences may be obtained from a variety of sources, including bacterial, fungal, viral, mammalian, or insect genes. (For example, see the regulatory sequences described in Goeddel, Gene Expression Technology: Methods in Enzymology 185, Academic Press, San Diego, Calif. (1990). Selection of appropriate regulatory sequences is dependent on the host cell chosen, and may be readily accomplished by one of ordinary skill in the art. Other sequences, such as an origin of replication, additional DNA restriction sites, enhancers, and sequences conferring inducibility of transcription may also be incorporated into the expression vector.

The recombinant expression vectors may also contain a selectable marker gene which facilitates the selection of transformed or transfected host cells. Suitable selectable marker genes are genes encoding proteins such as G418 and hygromycin which confer resistance to certain drugs, β-galactosidase, chloramphenicol acetyltransferase, firefly luciferase, or an immunoglobulin or portion thereof such as the Fc portion of an immunoglobulin preferably IgG. The selectable markers may be introduced on a separate vector from the nucleic acid of interest.

The recombinant expression vectors may also contain genes that encode a fusion portion which provides increased expression of the recombinant peptide; increased solubility of the recombinant peptide; and/or aid in the purification of the recombinant peptide by acting as a ligand in affinity purification. For example, a proteolytic cleavage site may be inserted in the recombinant peptide to allow separation of the recombinant peptide from the fusion portion after purification of the fusion protein. Examples of fusion expression vectors include pGEX (Amrad Corp., Melbourne, Australia), pMAL (New England Biolabs, Beverly, Mass.) and pRIT5 (Pharmacia, Piscataway, N.J.) which fuse glutathione S-transferase (GST), maltose E binding protein, or protein A, respectively, to the recombinant protein.

Recombinant expression vectors may be introduced into host cells to produce a transformant host cell. Transformant host cells include prokaryotic and eukaryotic cells which have been transformed or transfected with a recombinant expression vector of the invention. The terms “transformed with”, “transfected with”, “transformation” and “transfection” are intended to include the introduction of nucleic acid (e.g. a vector) into a cell by one of many techniques known in the art. For example, prokaryotic cells can be transformed with nucleic acid by electroporation or calcium-chloride mediated transformation. Nucleic acid can be introduced into mammalian cells using conventional techniques such as calcium phosphate or calcium chloride co-precipitation, DEAE-dextran-mediated transfection, lipofectin, electroporation or microinjection. Suitable methods for transforming and transfecting host cells may be found in Sambrook et al. (Molecular Cloning: A Laboratory Manual, 2nd Edition, Cold Spring Harbor Laboratory press (1989)), and other laboratory textbooks.

Suitable host cells include a wide variety of prokaryotic and eukaryotic host cells. For example, the peptides of the invention may be expressed in bacterial cells such as E. coli, insect cells (using baculovirus), yeast cells or mammalian cells. Other suitable host cells can be found in Goeddel, Gene Expression Technology: Methods in Enzymology 185, Academic Press, San Diego, Calif. (1991).

The peptides of the invention may be tyrosine phosphorylated using the method described in Reedijk et al. (The EMBO Journal 11(4):1365, 1992). For example, tyrosine phosphorylation may be induced by infecting bacteria harbouring a plasmid containing a nucleotide sequence encoding a peptide of the invention, with a λgt11 bacteriophage encoding the cytoplasmic domain of the Elk tyrosine kinase as a LacZ-Elk fusion. Bacteria containing the plasmid and bacteriophage as a lysogen are isolated. Following induction of the lysogen, the expressed peptide becomes phosphorylated by the Elk tyrosine kinase.

The peptides of the invention may be synthesized by conventional techniques. For example, the peptides may be synthesized by chemical synthesis using solid phase peptide synthesis. These methods employ either solid or solution phase synthesis methods (see for example, J. M. Stewart, and J. D. Young, Solid Phase Peptide Synthesis, 2^{nd} Ed., Pierce Chemical Co., Rockford Ill. (1984) and G. Barany and R. B. Merrifield, The Peptides: Analysis Synthesis, Biology editors E. Gross and J. Meienhofer Vol. 2 Academic Press, New York, 1980, pp. 3-254 for solid phase synthesis techniques; and M Bodansky, Principles fo Peptide Synthesis, Springer-Verlag, Berlin 1984, and E. Gross and J. Meienhofer, Eds., The Peptides: Analysis, Synthesis, Biologu, suprs, Vol 1, for classical solution synthesis). By way of example, the peptides may be synthesized using 9- fluorenyl methoxycarbonyl (Fmoc) solid phase chemistry with direct incorporation of phosphotyrosine as the N-fluorenylmethoxy-carbonyl-O-dimethyl phosphono-L-tyrosine derivative.

N-terminal or C-terminal fusion proteins comprising a peptide of the invention conjugated with other molecules may be prepared by fusing, through recombinant techniques, the N-terminal or C-terminal of the peptide, and the sequence of a selected protein or selectable marker with a desired biological function. The resultant fusion proteins contain the peptide fused to the selected protein or marker protein as described herein. Examples of proteins which may be used to prepare fusion proteins include immunoglobulins, glutathione-S-transferase (GST), hemagglutinin (HA), and truncated myc.

Cyclic derivatives of the peptides of the invention are also part of the present invention. Cyclization may allow the peptide to assume a more favorable conformation for association with molecules in complexes of the invention. Cyclization may be achieved using techniques known in the art. For example, disulfide bonds may be formed between two appropriately spaced components having free sulfhydryl groups, or an amide bond may be formed between an amino group of one component and a carboxyl group of another component. Cyclization may also be achieved using an azobenzene-containing amino acid as described by Ulysse, L., et al., J. Am. Chem. Soc. 1995, 117, 8466-8467. The side chains of Tyr and Asn may be linked to form cyclic peptides. The components that form the bonds may be side chains of amino acids, non-amino acid components or a combination of the two. In an embodiment of the invention, cyclic peptides are contemplated that have a beta-turn in the right position. Beta-turns may be introduced into the peptides of the invention by adding the amino acids Pro-Gly at the right position.

It may be desirable to produce a cyclic peptide that is more flexible than the cyclic peptides containing peptide bond linkages as described above. A more flexible peptide may be prepared by introducing cysteines at the right and left position of the peptide and forming a disulphide bridge between the two cysteines. The two cysteines are arranged so as not to deform the beta-sheet and turn. The peptide is more flexible as a result of the length of the disulfide linkage and the smaller number of hydrogen bonds in the beta-sheet portion. The relative flexibility of a cyclic peptide can be determined by molecular dynamics simulations. Peptide mimetics may be designed based on information obtained by systematic replacement of L-amino acids by D-amino acids, replacement of side chains with groups having different electronic properties, and by systematic replacement of peptide bonds with amide bond replacements. Local conformational constraints can also be introduced to determine conformational requirements for activity of a candidate peptide mimetic. The mimetics may include isosteric amide bonds, or D-amino acids to stabilize or promote reverse turn conformations and to help stabilize the molecule. Cyclic amino acid analogues may be used to constrain amino acid residues to particular conformational states. The mimetics can also include mimics of inhibitor peptide secondary structures. These structures can model the 3-dimensional orientation of amino acid residues into the known secondary conformations of proteins. Peptoids may also be used which are oligomers of N-substituted amino acids and can be used as motifs for the generation of chemically diverse libraries of novel molecules.

Combined with certain formulations, the subject PDZ inhibitory peptides can be effective intracellular agents. However, in order to increase the efficacy of such peptides, the PDZ inhibitory peptide can be provided a fusion peptide along with a second peptide which promotes “transcytosis”, e.g., uptake of the peptide by epithelial cells. To illustrate, the PDZ inhibitory peptide of the present invention can be provided as part of a fusion polypeptide with all or a fragment of the N-terminal domain of the HIV protein Tat, e.g., residues 1-72 of Tat or a smaller fragment thereof which can promote transcytosis. In other embodiments, the PDZ inhibitory peptide can be provided a fusion polypeptide with all or a portion of the antenopedia III protein.

To further illustrate, the PDZ inhibitory peptide (or peptidomimetic) can be provided as a chimeric peptide which includes a heterologous peptide sequence (“internalizing peptide”) which drives the translocation of an extracellular form of a PDZ inhibitory peptide sequence across a cell membrane in order to facilitate intracellular localization of the PDZ inhibitory peptide. In this regard, the therapeutic PDZ inhibitory binding sequence is one which is active intracellularly. The internalizing peptide, by itself, is capable of crossing a cellular membrane by, e.g., transcytosis, at a relatively high rate. The internalizing peptide is conjugated, e.g., as a fusion protein, to the PDZ inhibitory peptide. The resulting chimeric peptide is transported into cells at a higher rate relative to the activator polypeptide alone to thereby provide an means for enhancing its introduction into cells to which it is applied, e.g., to enhance topical applications of the PDZ inhibitory peptide.

In one embodiment, the internalizing peptide is derived from the Drosophila antennapedia protein, or homologs thereof. The 60 amino acid long long homeodomain of the homeo-protein antennapedia has been demonstrated to translocate through biological membranes and can facilitate the translocation of heterologous polypeptides to which it is couples. See for example Derossi et al. (1994) J Biol Chem 269:10444-10450; and Perez et al. (1992) J Cell Sci 102:717-722. Recently, it has been demonstrated that fragments as small as 16 amino acids long of this protein are sufficient to drive internalization. See Derossi et al. (1996) J Biol Chem 271:18188-18193.

The present invention contemplates a PDZ inhibitory peptide or peptidomimetic sequence as described herein, and at least a portion of the Antennapedia protein (or homolog thereof) sufficient to increase the transmembrane transport of the chimeric protein, relative to the PDZ inhibitory peptide or peptidomimetic, by a statistically significant amount.

Another example of an internalizing peptide is the HIV transactivator (TAT) protein. This protein appears to be divided into four domains (Kuppuswamy et al. (1989) Nucl. Acids Res. 17:3551-3561). Purified TAT protein is taken up by cells in tissue culture (Frankel and Pabo, (1989) Cell 55:1189-1193), and peptides, such as the fragment corresponding to residues 37 -62 of TAT, are rapidly taken up by cell in vitro (Green and Loewenstein, (1989) Cell 55:1179-1188). The highly basic region mediates internalization and targeting of the internalizing moiety to the nucleus (Ruben et al., (1989) J. Virol. 63:1-8.

Another exemplary transcellular polypeptide can be generated to include a sufficient portion of mastoparan (T. Higashijima et al., (1990) J. Biol. Chem. 265:14176) to increase the transmembrane transport of the chimeric protein.

While not wishing to be bound by any particular theory, it is noted that hydrophilic polypeptides may be also be physiologically transported across the membrane barriers by coupling or conjugating the polypeptide to a transportable peptide which is capable of crossing the membrane by receptor-mediated transcytosis. Suitable internalizing peptides of this type can be generated using all or a portion of, e.g., a histone, insulin, transferrin, basic albumin, prolactin and insulin-like growth factor I (IGF-I), insulin-like growth factor II (IGF-II) or other growth factors. For instance, it has been found that an insulin fragment, showing affinity for the insulin receptor on capillary cells, and being less effective than insulin in blood sugar reduction, is capable of transmembrane transport by receptor-mediated transcytosis and can therefor serve as an internalizing peptide for the subject transcellular peptides and peptidomimetics. Preferred growth factor-derived internalizing peptides include EGF (epidermal growth factor)-derived peptides, such as CMHIESLDSYTC and CMYIEALDKYAC; TGF- beta (transforming growth factor beta)-derived peptides; peptides derived from PDGF (platelet-derived growth factor) or PDGF-2; peptides derived from IGF-I (insulin-like growth factor) or IGF-II; and FGF (fibroblast growth factor)-derived peptides.

Another class of translocating/intemalizing peptides exhibits pH-dependent membrane binding. For an internalizing peptide that assumes a helical conformation at an acidic pH, the internalizing peptide acquires the property of amphiphilicity, e.g., it has both hydrophobic and hydrophilic interfaces. More specifically, within a pH range of approximately 5.0-5.5, an internalizing peptide forms an alpha-helical, amphiphilic structure that facilitates insertion of the moiety into a target membrane. An alpha-helix- inducing acidic pH environment may be found, for example, in the low pH environment present within cellular endosomes. Such internalizing peptides can be used to facilitate transport of PDZ inhibitory peptides and peptidomimetics, taken up by an endocytic mechanism, from endosomal compartments to the cytoplasm.

A preferred pH-dependent membrane-binding internalizing peptide includes a high percentage of helix-forming residues, such as glutamate, methionine, alanine and leucine. In addition, a preferred internalizing peptide sequence includes ionizable residues having pKa's within the range of pH 5-7, so that a sufficient uncharged membrane-binding domain will be present within the peptide at pH 5 to allow insertion into the target cell membrane.

A particularly preferred pH-dependent membrane-binding internalizing peptide in this regard is aa1-aa2-aa3-EAALA(EALA)4-EALEALAA-amide, which represents a modification of the peptide sequence of Subbarao et al. (Biochemistry 26:2964, 1987). Within this peptide sequence, the first amino acid residue (aa1) is preferably a unique residue, such as cysteine or lysine, that facilitates chemical conjugation of the internalizing peptide to a targeting protein conjugate. Amino acid residues 2-3 may be selected to modulate the affinity of the internalizing peptide for different membranes. For instance, if both residues 2 and 3 are lys or arg, the internalizing peptide will have the capacity to bind to membranes or patches of lipids having a negative surface charge. If residues 2-3 are neutral amino acids, the internalizing peptide will insert into neutral membranes.

Yet other preferred internalizing peptides include peptides of apo-lipoprotein A-1 and B; peptide toxins, such as melittin, bombolittin, delta hemolysin and the pardaxins; antibiotic peptides, such as alamethicin; peptide hormones, such as calcitonin, corticotrophin releasing factor, beta endorphin, glucagon, parathyroid hormone, pancreatic polypeptide; and peptides corresponding to signal sequences of numerous secreted proteins. In addition, exemplary internalizing peptides may be modified through attachment of substituents that enhance the alpha-helical character of the internalizing peptide at acidic pH.

Yet another class of internalizing peptides suitable for use within the present invention include hydrophobic domains that are “hidden” at physiological pH, but are exposed in the low pH environment of the target cell endosome. Upon pH-induced unfolding and exposure of the hydrophobic domain, the moiety binds to lipid bilayers and effects translocation of the covalently linked polypeptide into the cell cytoplasm. Such internalizing peptides may be modeled after sequences identified in, e.g., Pseudomonas exotoxin A, clathrin, or Diphtheria toxin.

Pore-forming proteins or peptides may also serve as internalizing peptides herein. Pore- forming proteins or peptides may be obtained or derived from, for example, C9 complement protein, cytolytic T-cell molecules or NK-cell molecules. These moieties are capable of forming ring-like structures in membranes, thereby allowing transport of attached polypeptide through the membrane and into the cell interior.

Mere membrane intercalation of an internalizing peptide may be sufficient for translocation of the PDZ inhibitory peptide or peptidomimetic, across cell membranes. However, translocation may be improved by attaching to the internalizing peptide a substrate for intracellular enzymes (i.e., an “accessory peptide”). It is preferred that an accessory peptide be attached to a portion(s) of the internalizing peptide that protrudes through the cell membrane to the cytoplasmic face. The accessory peptide may be advantageously attached to one terminus of a translocating/internalizing moiety or anchoring peptide. An accessory moiety of the present invention may contain one or more amino acid residues. In one embodiment, an accessory moiety may provide a substrate for cellular phosphorylation (for instance, the accessory peptide may contain a tyrosine residue).

An exemplary accessory moiety in this regard would be a peptide substrate for N- myristoyl transferase, such as GNAAAARR (Eubanks et al., in: Peptides, Chemistry and Biology, Garland Marshall (ed.), ESCOM, Leiden, 1988, pp. 566-69) In this construct, an internalizing peptide would be attached to the C-terminus of the accessory peptide, since the N-terminal glycine is critical for the accessory moiety's activity. This hybrid peptide, upon attachment to an E2 peptide or peptidomimetic at its C-terminus, is N-myristylated and further anchored to the target cell membrane, e.g., it serves to increase the local concentration of the peptide at the cell membrane.

To further illustrate use of an accessory peptide, a phosphorylatable accessory peptide is first covalently attached to the C-terminus of an internalizing peptide and then incorporated into a fusion protein with a PDZ inhibitory peptide or peptidomimetic. The peptide component of the fusion protein intercalates into the target cell plasma membrane and, as a result, the accessory peptide is translocated across the membrane and protrudes into the cytoplasm of the target cell. On the cytoplasmic side of the plasma membrane, the accessory peptide is phosphorylated by cellular kinases at neutral pH. Once phosphorylated, the accessory peptide acts to irreversibly anchor the fusion protein into the membrane. Localization to the cell surface membrane can enhance the translocation of the polypeptide into the cell cytoplasm.

Suitable accessory peptides include peptides that are kinase substrates, peptides that possess a single positive charge, and peptides that contain sequences which are glycosylated by membrane-bound glycotransferases. Accessory peptides that are glycosylated by membrane-bound glycotransferases may include the sequence x-NLT-x, where “x” may be another peptide, an amino acid, coupling agent or hydrophobic molecule, for example. When this hydrophobic tripeptide is incubated with microsomal vesicles, it crosses vesicular membranes, is glycosylated on the luminal side, and is entrapped within the vesicles due to its hydrophilicity (C. Hirschberg et al., (1987) Ann. Rev. Biochem. 56:63-87). Accessory peptides that contain the sequence x-NLT-x thus will enhance target cell retention of corresponding polypeptide.

In another embodiment of this aspect of the invention, an accessory peptide can be used to enhance interaction of the PDZ inhibitory peptide or peptidomimetic with the target cell. Exemplary accessory peptides in this regard include peptides derived from cell adhesion proteins containing the sequence “RGD”, or peptides derived from laminin containing the sequence CDPGYIGSRC. Extracellular matrix glycoproteins, such as fibronectin and laminin, bind to cell surfaces through receptor-mediated processes. A tripeptide sequence, RGD, has been identified as necessary for binding to cell surface receptors. This sequence is present in fibronectin, vitronectin, C3bi of complement, von-Willebrand factor, EGF receptor, transforming growth factor beta, collagen type I, lambda receptor of E. coli, fibrinogen and Sindbis coat protein (E. Ruoslahti, Ann. Rev. Biochem. 57:375-413, 1988). Cell surface receptors that recognize RGD sequences have been grouped into a superfamily of related proteins designated “integrins”. Binding of “RGD peptides” to cell surface integrins will promote cell-surface retention, and ultimately translocation, of the polypeptide.

As described above, the internalizing and accessory peptides can each, independently, be added to the PDZ inhibitory peptide or peptidomimetic by either chemical cross-linking or in the form of a fusion protein. In the instance of fusion proteins, unstructured polypeptide linkers can be included between each of the peptide moieties.

In general, the internalization peptide will be sufficient to also direct export of the polypeptide. However, where an accessory peptide is provided, such as an RGD sequence, it may be necessary to include a secretion signal sequence to direct export of the fusion protein from its host cell. In preferred embodiments, the secretion signal sequence is located at the extreme N-terminus, and is (optionally) flanked by a proteolytic site between the secretion signal and the rest of the fusion protein.

In an exemplary embodiment, a PDZ inhibitory peptide or peptidomimietic is engineered to include an integrin-binding RGD peptide/SV40 nuclear localization signal (see, for example Hart S L et al., 1994; J. Biol. Chem.,269:12468-12474), such as encoded by the nucleotide sequence provided in the Nde1-EcoR1 fragment: catatgggtggctgccgtggcgatatgttcggttgcggtgctcctccaaaaaagaagagaaag-gtagctggattc, which encodes the RGD/SV40 nucleotide sequence: MGGCRGDMFGCGAPP-KKKRKVAGF. In another embodiment, the protein can be engineered with the HIV-1 tat(l-72) polypeptide, e.g., as provided by the Nde1-EcoR1 fragment:catatggagccagtagatcctagactagagccc-tggaagcatccaggaagtcagcctaaaactgcttgtaccaattgctattgtaaaaagtgttgctttcattgccaagtttgtttcataaca aaagcccttggcatctcctatggcaggaagaagcggagacagcgacgaagacctcctcaaggcagtcagactcatcaagtttctc taagtaagcaaggattc, which encodes the HIV-1 tat(1-72) peptide sequence: MEPVDPRLEPWKHPGSQPKT-ACTNCYCKKCCFHCQVCFITKALGISYGRKKRRQRRRPPQGSQTHQVSLSKQ. In still another embodiment, the fusion protein includes the HSV-1 VP22 polypeptide (Elliott G., O'Hare P (1997) Cell, 88:223-233) provided by the Nde1-EcoR1 fragment:

cat atg acc tct cgc cgc tcc gtg aag tcg ggt ccg cgg gag gtt ccg cgc gat gag tac
gag gat ctg tac tac acc ccg tct tca ggt atg gcg agt ccc gat agt ccg cct gac acc
tcc cgc cgt ggc gcc cta cag aca cgc tcg cgc cag agg ggc gag gtc cgt ttc gtc
cag tac gac gag tcg gat tat gcc ctc tac ggg ggc tcg tca tcc gaa gac gac gaa
cac ccg gag gtc ccc cgg acg cgg cgt ccc gtt tcc ggg gcg gtt ttg tcc ggc ccg
ggg cct gcg cgg gcg cct ccg cca ccc gct ggg tcc gga ggg gcc gga cgc aca ccc
acc acc gcc ccc cgg gcc ccc cga acc cag cgg gtg gcg act aag gcc ccc gcg gcc
ccg gcg gcg gag acc acc cgc ggc agg aaa tcg gcc cag cca gaa tcc gcc gca ctc
cca gac gcc ccc gcg tcg acg gcg cca acc cga tcc aag aca ccc gcg cag ggg ctg
gcc aga aag ctg cac ttt agc acc gcc ccc cca aac ccc gac gcg cca tgg acc ccc
cgg gtg gcc ggc ttt aac aag cgc gtc ttc tgc gcc gcg gtc ggg cgc ctg gcg gcc
atg cat gcc cgg atg gcg gcg gtc cag ctc tgg gac atg tcg cgt ccg cgc aca gac
gaa gac ctc aac gaa ctc ctt ggc atc acc acc atc cgc gtg acg gtc tgc gag ggc
aaa aac ctg ctt cag cgc gcc aac gag ttg gtg aat cca gac gtg gtg cag gac gtc
gac gcg gcc acg gcg act cga ggg cgt tct gcg gcg tcg cgc ccc acc gag cga cct
cga gcc cca gcc cgc tcc gct tct cgc ccc aga cgg ccc gtc gag gaa ttc

which encodes the HSV-1 VP22 peptide having the sequence:

MTSRRSVKSGPREVPRDEYEDLYYTPSSGMASPDSPPDTSRRGALQ
TRSRQRGEVRFVQYDESDYALYGGSSSEDDEHPEVPRTRRPVSGAV
LSGPGPARAPPPPAGSGGAGRTPTTAPRAPRTGRVATKAPAAPAAE
TTRGRKSAQPESAALPDAPASTAPTRSKTPAQGLARKLHFSTAPPNP
DAPWTPRVAGFNKRVFCAAVGRLAAMHARMAAVQLWDMSRPRT
DEDLNELLGITTIRVTVCEGKNLLQRANELVNPDVVQDVDAATATR
GRSAASRPTERPRAPARSASRPRRPVE

In still another embodiment, the fusion protein includes the C-terminal domain of the VP22 protein from, e.g., the nucleotide sequence (Nde1-EcoR1 fragment):

cat atg gac gtc gac gcg gcc acg gcg act cga ggg cgt tct gcg gcg tcg cgc ccc
acc gag cga cct cga gcc cca gcc cgc tcc gct tct cgc ccc aga cgg ccc gtc gag
gaa ttc

which encodes the VP22 (C-terminal domain) peptide sequence: MDVDAATATRGRSA-ASRPTERPRAPARSASRPRRPVE

In certain instances, it may also be desirable to include a nuclear localization signal as part of the PDZ inhibitory peptide.

In the generation of fusion polypeptides including the subject PDZ inhibitory peptides, it may be necessary to include unstructured linkers in order to ensure proper folding of the various peptide domains. Many synthetic and natural linkers are known in the art and can be adapted for use in the present invention, including the (Gly₃Ser)₄ linker.

PDZ Inhibitory Mimetics

In other embodiments, the subject PDZ inhibitory therapeutics are peptidomimetics of the PDZ inhibitory peptide. Peptidomimetics are compounds based on, or derived from, peptides and proteins. The PDZ inhibitory peptidomimetics of the present invention typically can be obtained by structural modification of a known PDZ inhibitory peptide sequence using unnatural amino acids, conformational restraints, isosteric replacement, and the like. The subject peptidomimetics constitute the continum of structural space between peptides and non-peptide synthetic structures; PDZ inhibitory peptidomimetics may be useful, therefore, in delineating pharmacophores and in helping to translate peptides into nonpeptide compounds with the activity of the parent PDZ inhibitory peptides.

Moreover, as is apparent from the present disclosure, mimetopes of the subject PDZ inhibitory peptides can be provided. Such peptidomimetics can have such attributes as being non-hydrolyzable (e.g., increased stability against proteases or other physiological conditions which degrade the corresponding peptide), increased specificity and/or potency, and increased cell permeability for intracellular localization of the peptidomimetic. For illustrative purposes, peptide analogs of the present invention can be generated using, for example, benzodiazepines (e.g., see Freidinger et al. in Peptides: Chemistry and Biology, G. R. Marshall ed., ESCOM Publisher: Leiden, Netherlands, 1988), substituted gama lactam rings (Garvey et al. in Peptides: Chemistry and Biology, G. R. Marshall ed., ESCOM Publisher: Leiden, Netherlands, 1988, p123), C-7 mimics (Huffman et al. in Peptides: Chemistry and Biologyy, G. R. Marshall ed., ESCOM Publisher: Leiden, Netherlands, 1988, p. 105), keto-methylene pseudopeptides (Ewenson et al. (1986) J Med Chem 29:295; and Ewenson et al. in Peptides: Structure and Function (Proceedings of the 9th American Peptide Symposium) Pierce Chemical Co. Rockland, Ill., 1985), β-turn dipeptide cores (Nagai et al. (1985) Tetrahedron Lett 26:647; and Sato et al. (1986) J Chem Soc Perkin Trans 1:1231), -aminoalcohols (Gordon et al. (1985) Biochem Biophys Res Commun126:419; and Dann et al. (1986) Biochem Biophys Res Commun 134:71), diaminoketones (Natarajan et al. (1984) Biochem Biophys Res Commun 124:141), and methyleneamino-modifed (Roark et al. in Peptides: Chemistry and Biology, G. R. Marshall ed., ESCOM Publisher: Leiden, Netherlands, 1988, p134). Also, see generally, Session III: Analytic and synthetic methods, in in Peptides: Chemistry and Biology, G. R. Marshall ed., ESCOM Publisher: Leiden, Netherlands, 1988)

In addition to a variety of sidechain replacements which can be carried out to generate the subject PDZ inhibitory peptidomimetics, the present invention specifically contemplates the use of conformationally restrained mimics of peptide secondary structure. Numerous surrogates have been developed for the amide bond of peptides. Frequently exploited surrogates for the amide bond include the following groups (i) trans-olefins, (ii) fluoroalkene, (iii) methyleneamino, (iv) phosphonamides, and (v) sulfonamides.

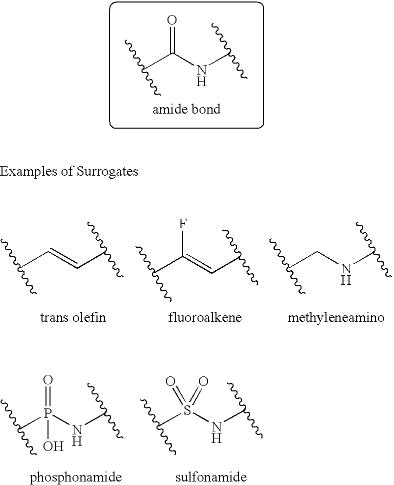

Additionally, peptidomimietics based on more substantial modifications of the backbone of the PDZ inhibitory peptide can be used. Peptidomimetics which fall in this category include (i) retro-inverso analogs, and (ii) N-alkyl glycine analogs (so-called peptoids).

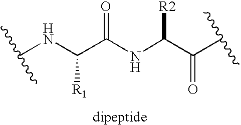

Examples of Analogs

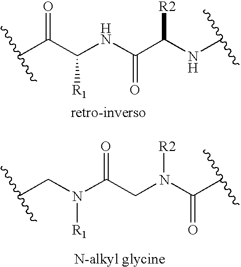

Furthermore, the methods of combinatorial chemistry are being brought to bear, c.f. Verdine et al. PCT publication WO9948897, on the development of new peptidomimetics. For example, one embodiment of a so-called “peptide morphing” strategy focuses on the random generation of a library of peptide analogs that comprise a wide range of peptide bond substitutes.

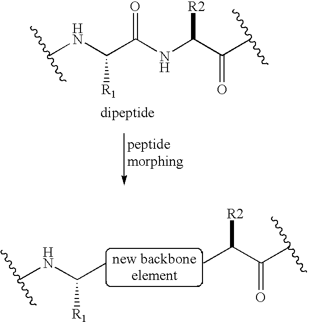

In an exemplary embodiment, the peptidomimetic can be derived as a retro-inverso analog of the peptide

Retro-inverso analogs can be made according to the methods known in the art, such as that described by the Sisto et al. U.S. Pat. No. 4,522,752. As a general guide, sites which are most susceptible to proteolysis are typically altered, with less susceptible amide linkages being optional for mimetic switching The final product, or intermediates thereof, can be purified by HPLC.

In another illustrative embodiment, the peptidomimetic can be derived as a retro-enatio analog of the peptide. Retro-enantio analogs such as this can be synthesized commercially available D-amino acids (or analogs thereof) and standard solid- or solution-phase peptide-synthesis techniques. For example, in a preferred solid-phase synthesis method, a suitably amino-protected (t-butyloxycarbonyl, Boc) residue (or analog thereof) is covalently bound to a solid support such as chloromethyl resin. The resin is washed with dichloromethane (DCM), and the BOC protecting group removed by treatment with TFA in DCM. The resin is washed and neutralized, and the next Boc-protected D-amino acid is introduced by coupling with diisopropylcarbodiimide. The resin is again washed, and the cycle repeated for each of the remaining amino acids in turn. When synthesis of the protected retro-enantio peptide is complete, the protecting groups are removed and the peptide cleaved from the solid support by treatment with hydrofluoric acid/anisole/dimethyl sulfide/thioanisole. The final product is purified by HPLC to yield the pure retro-enantio analog.

In still another illustrative embodiment, trans-olefin derivatives can be made for any of the subject polypeptides. A trans olefin analog of PDZ inhibitory peptide can be synthesized according to the method of Y. K. Shue et al. (1987) Tetrahedron Letters 28:3225 and also according to other methods known in the art. It will be appreciated that variations in the cited procedure, or other procedures available, may be necessary according to the nature of the reagent used. It is further possible couple the pseudodipeptides synthesized by the above method to other pseudodipeptides, to make peptide analogs with several olefinic functionalities in place of amide functionalities.

Still another class of peptidomimetic derivatives include phosphonate derivatives. The synthesis of such phosphonate derivatives can be adapted from known synthesis schemes. See, for example, Loots et al. in Peptides: Chemistry and Biology, (Escom Science Publishers, Leiden, 1988, p. 118); Petrillo et al. in Peptides: Structure and Function (Proceedings of the 9th American Peptide Symposium, Pierce Chemical Co. Rockland, Ill., 1985).

Many other peptidomimetic structures are known in the art and can be readily adapted for use in the the subject PDZ inhibitory peptidomimetics. To illustrate, the PDZ inhibitory peptidomimetic may incorporate the 1-azabicyclo[4.3.0]nonane surrogate ( see Kim et al. (1997) J. Org. Chem. 62:2847), or an N-acyl piperazic acid (see Xi et al. (1998) J. Am. Chem. Soc. 120:80), or a 2-substituted piperazine moiety as a constrained amino acid analogue (see Williams et al. (1996) J. Med. Chem. 39:1345-1348). In still other embodiments, certain amino acid residues can be replaced with aryl and bi-aryl moieties, e.g., monocyclic or bicyclic aromatic or heteroaromatic nucleus, or a biaromatic, aromatic-heteroaromatic, or biheteroaromatic nucleus.

The subject PDZ inhibitory peptidomimetics can be optimized by, e.g., combinatorial synthesis techniques combined with such high throughput screening as described herein.

Moreover, other examples of mimetopes include, but are not limited to, protein-based compounds, carbohydrate-based compounds, lipid-based compounds, nucleic acid-based compounds, natural organic compounds, synthetically derived organic compounds, anti-idiotypic antibodies and/or catalytic antibodies, or fragments thereof. A mimetope can be obtained by, for example, screening libraries of natural and synthetic compounds for compounds capable of binding to the PDZ inhibitory domain or inhibiting the interaction between the PDZ inhibitory domain and the natural ligand. A mimetope can also be obtained, for example, from libraries of natural and synthetic compounds, in particular, chemical or combinatorial libraries (i.e., libraries of compounds that differ in sequence or size but that have the same building blocks). A mimetope can also be obtained by, for example, rational drug design. In a rational drug design procedure, the three-dimensional structure of a compound of the present invention can be analyzed by, for example, nuclear magnetic resonance (NMR) or x-ray crystallography. The three-dimensional structure can then be used to predict structures of potential mimetopes by, for example, computer modelling. the predicted mimetope structures can then be produced by, for example, chemical synthesis, recombinant DNA technology, or by isolating a mimetope from a natural source (e.g., plants, animals, bacteria and fungi).

Peptides that interact with the molecules in a complex of the invention may be developed using a biological expression system. The use of these systems allows the production of large libraries of random peptide sequences and the screening of these libraries for peptide sequences that bind to particular proteins. Libraries may be produced by cloning synthetic DNA that encodes random peptide sequences into appropriate expression vectors. (see Christian et al 1992, J. Mol. Biol. 227:711; Devlin et al, 1990 Science 249:404; Cwirla et al 1990, Proc. Natl. Acad, Sci. USA, 87:6378). Libraries may also be constructed by concurrent synthesis of overlapping peptides (see U.S. Pat. No. 4,708,871).

Peptides of the invention may be used to identify lead compounds for drug development. The structure of the peptides described herein can be readily determined by a number of methods such as NMR and X-ray crystallography. A comparison of the structures of peptides similar in sequence, but differing in the biological activities they elicit in target molecules can provide information about the structure-activity relationship of the target. Information obtained from the examination of structure-activity relationships can be used to design either modified peptides, or other small molecules or lead compounds which can be tested for predicted properties as related to the target molecule. The activity of the lead compounds can be evaluated using assays similar to those described herein.

Information about structure-activity relationships may also be obtained from co-crystallization studies. In these studies, a peptide with a desired activity is crystallized in association with a target molecule, and the X-ray structure of the complex is determined. The structure can then be compared to the structure of the target molecule in its native state, and information from such a comparison may be used to design compounds expected to possess desired activities.

The peptides of the invention may be converted into pharmaceutical salts by reacting with inorganic acids such as hydrochloric acid, sulfuric acid, hydrobromic acid, phosphoric acid, etc., or organic acids such as formic acid, acetic acid, propionic acid, glycolic acid, lactic acid, pyruvic acid, oxalic acid, succinic acid, malic acid, tartaric acid, citric acid, benzoic acid, salicylic acid, benezenesulfonic acid, and toluenesulfonic acids.

The peptides of the invention may be used to prepare antibodies. Conventional methods can be used to prepare the antibodies.

The peptides and antibodies specific for the peptides of the invention may be labelled using conventional methods with various enzymes, fluorescent materials, luminescent materials and radioactive materials. Suitable enzymes, fluorescent materials, luminescent materials, and radioactive material are well known to the skilled artisan. Antibodies and labeled antibodies specific for the peptides of the invention may be used to screen for proteins containing PDZ domain binding sites.

Computer modelling techniques known in the art may also be used to observe the interaction of a peptide of the invention, and truncations and analogs thereof with a molecule in a complex of the invention e.g. PDZ domain containing protein (for example, Homology Insight II and Discovery available from BioSym/Molecular Simulations, San Diego, Calif., U.S.A.). If computer modelling indicates a strong interaction, the peptide can be synthesized and tested for its ability to interfere with the binding of the molecules of a complex discussed herein.

Methods for Identifying or Evaluating Substances/Compounds

The methods described herein are designed to identify substances and compounds that modulate the activity of a complex of the invention thus potentially affecting cellular processes associated with B class ephrins and/or PDZ domain containing proteins. Novel substances are therefore contemplated that bind to molecules in the complexes, or bind to other proteins that interact with the molecules, to compounds that interfere with, or enhance the interaction of the molecules in a complex, or other proteins that interact with the molecules.

The substances and compounds identified using the methods of the invention include but are not limited to peptides such as soluble peptides including Ig-tailed fusion peptides, members of random peptide libraries and combinatorial chemistry-derived molecular libraries made of D- and/or L-configuration amino acids, phosphopeptides (including members of random or partially degenerate, directed phosphopeptide libraries), antibodies [e.g. polyclonal, monoclonal, humanized, anti-idiotypic, chimeric, single chain antibodies, fragments, (e.g. Fab, F(ab)₂, and Fab expression library fragments, and epitope-binding fragments thereof)], and small organic or inorganic molecules. The substance or compound may be an endogenous physiological compound or it may be a natural or synthetic compound.

Substances which modulate the activity of a complex of the invention can be identified based on their ability to bind to a molecule in the complex. Therefore, the invention also provides methods for identifying novel substances which bind molecules in the complex. Substances identified using the methods of the invention may be isolated, cloned and sequenced using conventional techniques.

Novel substances which can bind with a molecule in a complex of the invention may be identified by reacting one of the molecules with a test substance which potentially binds to the molecule, under conditions which permit binding of the molecule and test substance, and detecting binding. Binding may be detected by assaying for substance-molecule conjugates, for free substance, or for non-complexed molecules, or activation of the molecule. Conditions which permit the formation of substance-molecule conjugates may be selected having regard to factors such as the nature and amounts of the substance and the molecule.

The substance-molecule conjugate, free substance or non-complexed molecules may be isolated by conventional isolation techniques, for example, salting out, chromatography, electrophoresis, gel filtration, fractionation, absorption, polyacrylamide gel electrophoresis, agglutination, or combinations thereof. To facilitate the assay of the components, antibody against the molecule or the substance, or labeled molecule, or a labeled substance may be utilized. The antibodies, proteins, or substances may be labeled with a detectable substance as described above.

Activation may be assayed by measuring phosphorylation of a molecule, binding of receptors or cellular proteins to a molecule, or in a cellular assay, by assaying for a biological affect on the cell, such as inhibition or stimulation of proliferation, differentiation or migration.

A molecule, or complex of the invention, or the substance used in the method of the invention may be insolubilized. For example, a molecule, or substance may be bound to a suitable carrier such as agarose, cellulose, dextran, Sephadex, Sepharose, carboxymethyl cellulose polystyrene, filter paper, ion-exchange resin, plastic film, plastic tube, glass beads, polyamine-methyl vinyl-ether-maleic acid copolymer, amino acid copolymer, ethylene-maleic acid copolymer, nylon, silk, etc. The carrier may be in the shape of, for example, a tube, test plate, beads, disc, sphere etc. The insolubilized protein or substance may be prepared by reacting the material with a suitable insoluble carrier using known chemical or physical methods, for example, cyanogen bromide coupling.

The invention also contemplates a method for evaluating a compound for its ability to modulate the biological activity of a complex of the invention, by assaying for an agonist or antagonist of the binding of the molecules in the complex. The basic method for evaluating if a compound is an agonist or antagonist of the binding of molecules in a complex of the invention, is to prepare a reaction mixture containing the molecules and the test compound under conditions which permit the molecules to bind and form a complex. The test compound may be initially added to the mixture, or may be added subsequent to the addition of molecules. Control reaction mixtures without the test compound or with a placebo are also prepared. The formation of complexes is detected and the formation of complexes in the control reaction but not in the reaction mixture indicates that the test compound interferes with the interaction of the molecules. Increased complex formation relative to a control reaction indicates that the test compound enhances the interaction of the molecules. The reactions may be carried out in the liquid phase or the molecules, or test compound may be immobilized as described herein.

It will be understood that the agonists and antagonists that can be assayed using the methods of the invention may act on one or more of the binding sites on the interacting molecules in the complex including agonist binding sites, competitive antagonist binding sites, non-competitive antagonist binding sites or allosteric sites.

The invention also makes it possible to screen for antagonists that inhibit the effects of an agonist of the interaction of molecules in a complex of the invention. Thus, the invention may be used to assay for a compound that competes for the same binding site of a molecule in a complex of the invention.

The invention also contemplates methods for identifying novel compounds that bind to proteins that interact with a molecule of a complex of the invention. Protein-protein interactions may be identified using conventional methods such as co-immunoprecipitation, crosslinking and co-purification through gradients or chromatographic columns. Methods may also be employed that result in the simultaneous identification of genes which encode proteins interacting with a molecule. These methods include probing expression libraries with labeled molecules. Additionally, x-ray crystallographic studies may be used as a means of evaluating interactions with substances and molecules. For example, purified recombinant molecules in a complex of the invention when crystallized in a suitable form are amenable to detection of intra-molecular interactions by x-ray crystallography. Spectroscopy may also be used to detect interactions and in particular, a quadrupole/time-of-flight hybrid instrument (QqTOF) may be used.

Two-hybrid systems may also be used to detect protein interactions in vivo. Generally, plasmids are constructed that encode two hybrid proteins. A first hybrid protein consists of the DNA-binding domain of a transcription activator protein fused to a molecule in a complex of the invention, and the second hybrid protein consists of the transcription activator protein's activator domain fused to an unkown protein encoded by a cDNA which has been recombined into the plasmid as part of a cDNA library. The plasmids are transformed into a strain of yeast (e.g. S. cerevisiae) that contains a reporter gene (e.g. lacZ, luciferase, alkaline phosphatase, horseradish peroxidase) whose regulatory region contains the transcription activator's binding site. The hybrid proteins alone cannot activate the transcription of the reporter gene. However, interaction of the two hybrid proteins reconstitutes the functional activator protein and results in expression of the reporter gene, which is detected by an assay for the reporter gene product.

It will be appreciated that fusion proteins and recombinant fusion proteins may be used in the above-described methods. It will also be appreciated that the complexes of the invention may be reconstituted in vitro using recombinant molecules and the effect of a test substance may be evaluated in the reconstituted system.

The reagents suitable for applying the methods of the invention to evaluate substances and compounds may be packaged into convenient kits providing the necessary materials packaged into suitable containers. The kits may also include suitable supports useful in performing the methods of the invention.

### Compositions and Treatments

The complexes, peptides, and antibodies of the invention, and substances and compounds identified using the methods of the invention may be used to modulate cellular processes such as proliferation, growth, and/or differentiation of cells associated with B class ephrins and/or PDZ domain containing proteins (in particular axonogenesis, nerve cell interactions and regeneration of the nervous system). Therefore they may be used to treat conditions in a subject in which the compounds or substances are introduced. Thus, the substances may be used for the treatment of disorders associated with a B class ephrin such as disorders of the nervous system including neurodegenerative diseases and cases of nerve injury.

Accordingly, the complexes, peptides, substances, antibodies, and compounds may be formulated into pharmaceutical compositions for administration to subjects in a biologically compatible form suitable for administration in vivo. By “biologically compatible form suitable for administration in vivo” is meant a form of the substance to be administered in which any toxic effects are outweighed by the therapeutic effects. The substances may be administered to living organisms including humans, and animals. Administration of a “therapeutically active amount” of the pharmaceutical compositions of the present invention is defined as an amount effective, at dosages and for periods of time necessary to achieve the desired result. For example, a therapeutically active amount of a substance may vary according to factors such as the disease state, age, sex, and weight of the individual, and the ability of antibody to elicit a desired response in the individual. Dosage regima may be adjusted to provide the optimum therapeutic response. For example, several divided doses may be administered daily or the dose may be proportionally reduced as indicated by the exigencies of the therapeutic situation.

The active substance may be administered in a convenient manner such as by injection (subcutaneous, intravenous, etc.), oral administration, inhalation, transdermal application, or rectal administration. Depending on the route of administration, the active substance may be coated in a material to protect the compound from the action of enzymes, acids and other natural conditions that may inactivate the compound.

The compositions described herein can be prepared by per se known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985). On this basis, the compositions include, albeit not exclusively, solutions of the substances or compounds in association with one or more pharmaceutically acceptable vehicles or diluents, and contained in buffered solutions with a suitable pH and iso-osmotic with the physiological fluids.

The activity of the complexes, substances, compounds, antibodies, and compositions of the invention may be confirmed in animal experimental model systems.

The invention also provides methods for studying the function of a complex of the invention. Cells, tissues, and non-human animals lacking in the complexes or partially lacking in molecules in the complexes may be developed using recombinant expression vectors of the invention having specific deletion or insertion mutations in the molecules. A recombinant expression vector may be used to inactivate or alter the endogenous gene by homologous recombination, and thereby create complex deficient cells, tissues or animals. Null alleles may be generated in cells and may then be used to generate transgenic non-human animals.

The following non-limiting example is illustrative of the present invention:

### EXAMPLE

### Experimental Procedures

Peptide synthesis

The B ephrin C-terminal peptide probe of sequence biotin-Aca-GPPQSPPNIpYYKV (SEQ. ID. NO.6), related peptides NIpYpYKV (SEQ. ID. NO. 7), NIpYYKV (SEQ. ID. NO. 8), NIYpYKV (SEQ. ID. NO. 9), NIYYKV (SEQ. ID. NO. 10), and DHQpYpYND (SEQ. ID. NO. 11), were synthesized as described previously (29).

Isolation of PDZ Domain-encoding cDNA Clones

A λEXlox 10.5 day mouse embryo expression library (Novagen) was plated at an initial density of 10,000 plaque-forming units/15 cm petri plate. Library screening was performed using a biotinylated peptide probe conjugated to streptavidin-alkaline phosphatase following a procedure similar to that described by Sparks et al. (30). To isolate more coding sequence for PHIP, an EcoRI/Pst I fragment of PHIP cDNA (encoding amino acid residues 462-602) was radiolabelled with [α-³²P]dCTP and used to screen the λEXIox 10.5 day mouse embryo library. The DNA sequencing of positive clones was carried out using the ALF automated DNA sequencer (Amersham Pharmacia Biotech).

Antibodies, Constructs and Mutagenesis

Anti-ligand antibodies (Santa Cruz) were raised against residues 329-346 of hEphrin B1. Anti-FLAG M2 monoclonal antibodies were purchased from Eastman Kodak Company. The expression construct of ephrin B1 cDNA in vector pJFE14 has been described (18). Full-length syntenin cDNA was subcloned in frame into the mammalian expression vector pFLAG CMV2 (Eastman Kodak) using standard cloning procedures. For GST fusion constructs, cDNA sequences of syntenin (full length: residues 1-299; PDZ 1+2: residues 101-299; PDZ1: residues 101-211; PDZ2: residues 172-299) were cloned into pGEX4T2 (Amersham Pharmacia Biotech). FAP-1 (Fas associated phosphatase) PDZ3 and FAP-1 PDZ5 constructs have been described (1). The ephrin B1 Val deletion mutation was constructed by the removal of nucleotides coding for the C-terminal V346 using a PCR-mediated protocol. The PpuMI/EcoRI PCR fragment carrying the mutated region was subcloned into the full-length ephrin B1 cDNA in pJFE14. This mutation and all fusion constructs were confirmed by sequencing of both strands of the affected region.

Immunoprecipitation and Western Blot Analysis

Cos-1 cells were maintained in DMEM supplemented with 10% fetal bovine serum (FBS). Transient transfections were performed using Lipofectin reagent and Opti-MEM medium (Life Technologies Inc.) as outlined by the manufacturer. To reduce phosphorylation of ephrin B1 by binding to endogenously expressed EphB receptors or by stimulation with serum growth factors, transfected cells were transferred from 10 cm to 15 cm plates 24 h after transfection and serum starved in DMEM 0.5% FBS 12 h prior to cell lysis. Transfected cells were rinsed once in PBSA and lysed in PLC lysis buffer (5) with 10 μg/ml aprotonin, 10 μg/ml leupeptin, 1 mM sodium vanadate and 1 mM phenylmethylsulfonyl fluoride added. Immunoprecipitations were performed for 1 h at 4° C. using 1 μg anti-ephrin B1 antibody or 1 μg anti-IL-3 receptor a antibody with protein A-sepharose. GST mixing experiments were carried out by 1 h incubation at 4° C. of lysate with 5-10 μg of fusion protein immobilized on glutathione sepharose. For the peptide competition experiments, peptides were included in the incubation with the GST fision proteins at a final concentration of 100 μM. Beads for both immunoprecipitations and GST mixing experiments were washed 2-3 times in HNTG buffer (5). Proteins were separated by 10% SDS-PAGE, transferred to Immobilon-P membrane (Millipore) and immunoblotted with the appropriate antibody. Blots were developed by Enhanced Chemiluminescence (Pierce).

Fluorescence Polarization Analysis

Binding constant determination and peptide competition studies were carried out using fluorescence polarization on a Beacon 2000 Fluorescence Polarization System (Pan Vera, Wis.) equipped with a 100-μl sample chamber. Fluorescein-labeled probes were prepared through reaction of B ephrin C-terminal peptides with 5-(and-6-)-carboxyfluorescein, succinimidyl ester (Molecular Probes, Oreg.) and purified by reverse-phase HPLC. The authenticity of the fluorescein-labelled peptides were confirmed by mass spectroscopy. In the binding studies, the fluorescein-labelled peptide probe was dissolved in 20 mM phosphate pH 7.0, 100 mM NaCl, and 2 mM DTT to a concentration of 25 nM and a known quantity of GST- fusion protein added. The reaction mixtures were allowed to stand for 10 min at room temperature prior to each measurement. All fluorescence polarization measurements were conducted at 22° C.

### Results

Identification of potential binding partners for the putative PDZ binding site of B ephrins

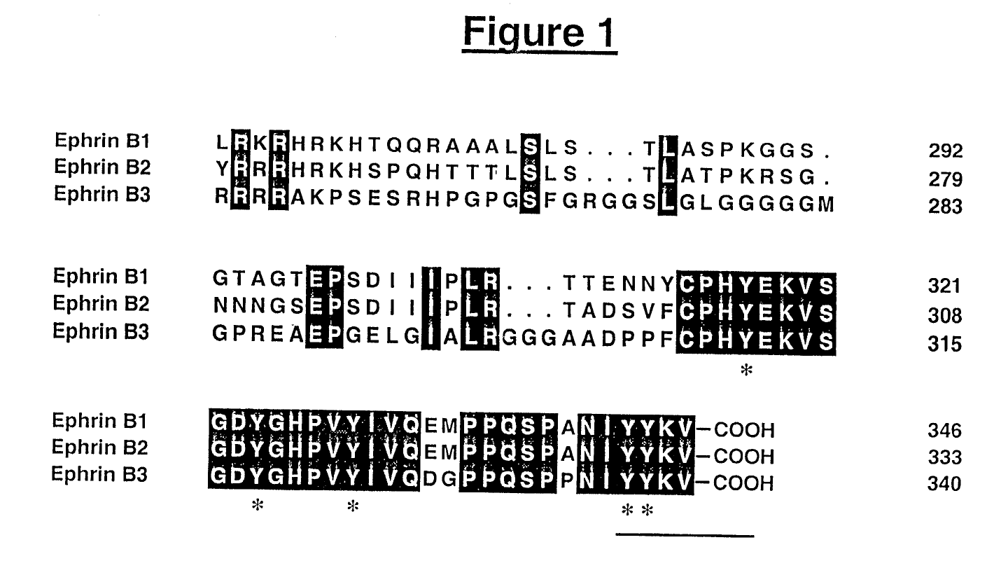

As one approach towards identifying proteins that interact with the cytoplasmic tails of B-type ephrins, the C-terminal regions of the transmembrane ephrins were initially examined for conserved peptide motifs that might bind modular domains of intracellular signaling proteins. The extreme carboxy terminus of the three known B ephrins has a conserved sequence reminiscent of known or predicted binding sites for PDZ domains (FIG. 1). Two strategies were employed to identify PDZ domain-containing proteins with the potential to recognize the B ephrins. Firstly, comparison of the known binding specificities of PDZ domains, predicted through the use of an oriented peptide library technique, revealed the fifth PDZ domain of the cytoplasmic tyrosine phosphatase FAP-1 (Fas-associated phosphatase) as a possible ephrin B binding partner (FIG. 2A). FAP-1 (also known as PTP-bas and PTP-L1) has at least six PDZ domains, an element related to the Band 4.1 cytoskeletal polypeptide, and a C-terminal tyrosine phosphatase domain (31-33). The fifth PDZ domain binds in vitro to peptides with the consensus E-(I/Y/V)-Y-(Y/K)-(V/K/I), which closely matches the conserved C-terminus of B-type ephrins (YYKV) (1).

A more direct approach to isolate ephrin B-binding proteins was undertaken by screening a cDNA expression library from a day 10.5 mouse embryo with a peptide probe based on the putative PDZ domain binding site of ephrin B3. The probe was a biotinylated peptide, biotin-Aca-GPPQSPPNIpYYKV (SEQ. ID. No. 6), conjugated to streptavidin-alkaline phosphatase. Although this peptide contained a phosphotyrosine residue at the -3 position relative to the C-terminal valine, it was anticipitated that the alkaline phosphatase used in the screen would at least partially dephosphorylate the probe, allowing detection of both tyrosine phosphorylation dependent and independent binding. The screening of approximately 500,000 cDNA clones yielded four distinct cDNA products that bound to the ephrin B3 C-terminal peptide, of which three were subsequently found to contain PDZ domains upon sequence analysis (FIGS. 2B and 2C). One of these cDNAs encodes a portion of the adaptor protein GRIP, from the sixth PDZ domain to the carboxy terminus (amino acid residues 642-1112). GRIP is an 180 kDa protein composed of seven PDZ domains, originally identified by its ability to bind the C-terminus of AMPA receptors through PDZ domains 4 and 5 (34). A second cDNA isolated by this approach contained the entire coding sequence for the PDZ domain-containing protein syntenin. Syntenin was first reported as a transcript down-regulated during melanoma differentiation (termed Mda-9) and subsequently shown to interact via its two PDZ domains with the C-terminus of the transmembrane syndecan proteins (35,36). A third clone identified in this screen was a partial cDNA encoding the carboxy-terminal fragment of a novel PDZ domain-containing protein (termed PHIP for ephrin interacting protein). Analysis of the sequence of the PHIP cDNA fragment revealed the presence of two adjacent PDZ domains followed by a 50 amino acid C-terminal stretch. The PHIP cDNA fragment was subsequently used as a probe to isolate a transcript from a day 10.5 mouse embryo library. The predicted sequence of PHIP indicates that it encodes a total of three PDZ domains and is closely related to PAR-3, a C. elegans protein involved in regulating polarity of the early embryo (FIG. 2D) (37). Of these candidates, FAP-1 PDZ5 and syntenin were further investigated for their binding to B ephrins.

Syntenin and FAP-1 PDZ5 Bind Ephrin B1 In vitro

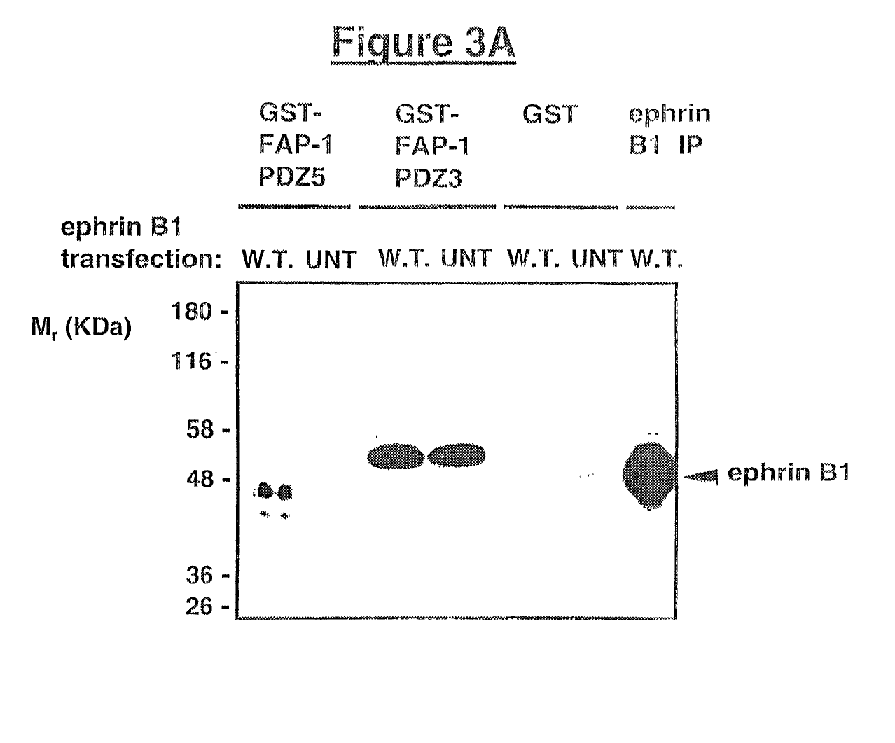

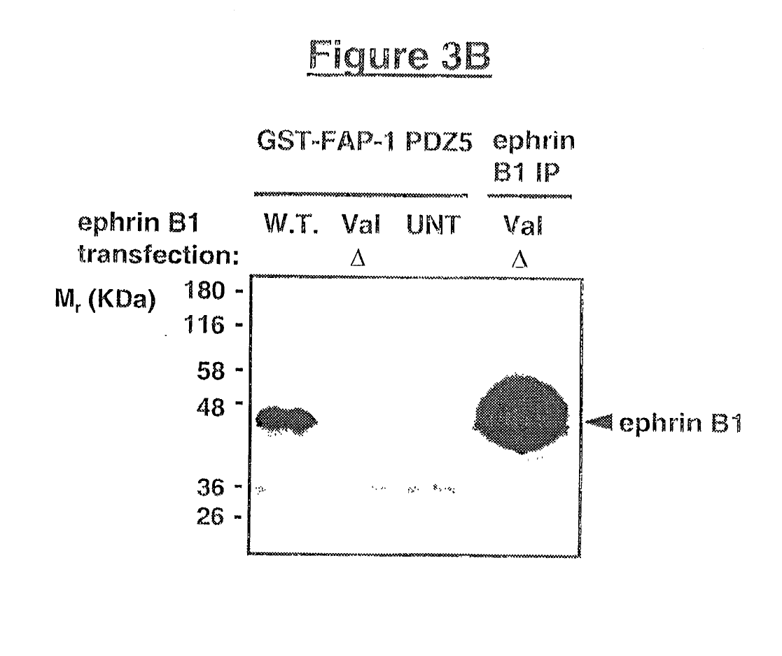

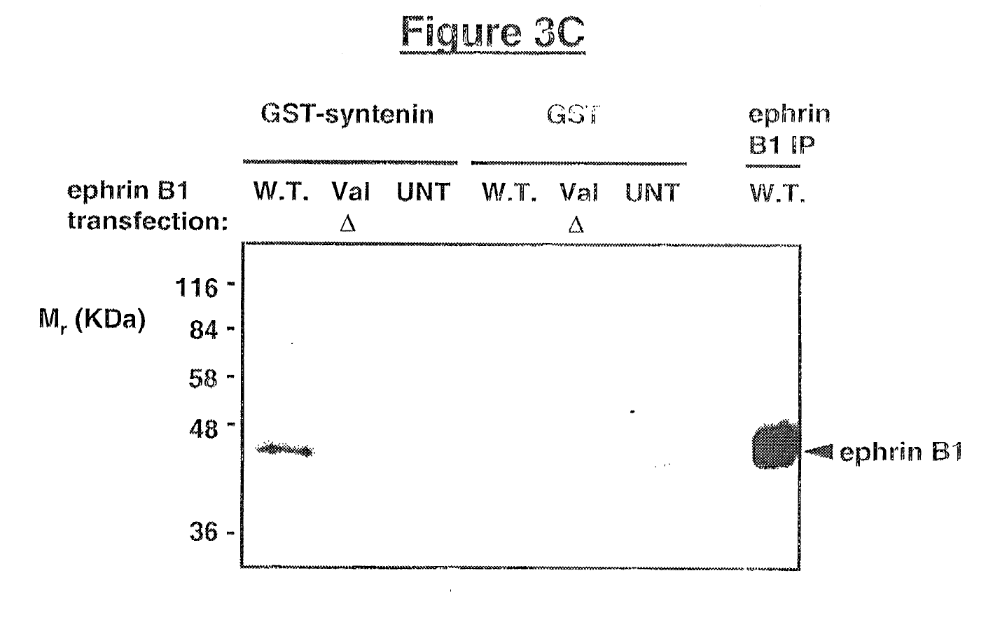

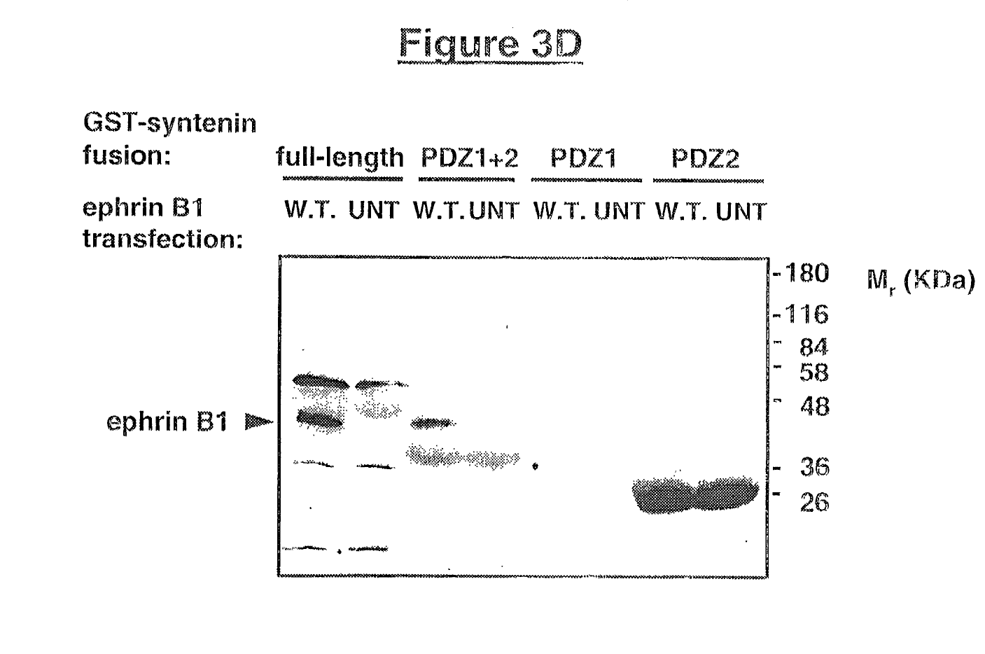

To determine if either syntenin or FAP-1 could interact with ephrin B1 in vitro, GST-fusions containing the fifth PDZ domain of FAP-1 or full-length syntenin were incubated with lysates of ephrin B1-transfected Cos-1 cells. Recovery of these immobilized GST fusion proteins and immuno-blotting of associated proteins with anti-ephrin B1 antibody revealed that both FAP-1 PDZ5 and full-length syntenin were able to bind intact ephrin B1 (FIGS. 3A and 3C). The region of syntenin required for binding to ephrin B1 was mapped using GST fusions containing defined fragments of the syntenin protein. The minimal sequence necessary for a strong interaction included both PDZ domains of syntenin but not the amino-terminal third of the protein (FIG. 3D). Interestingly, both PDZ domains of syntenin are also required for binding to the C-terminal sequence of syndecans, suggesting that the involvment of two PDZ domains in the binding of a single target site may be a common feature of syntenin interactions (36). While the syntenin PDZ1 domain alone was unable to associate with ephrin B1, the second PDZ domain of syntenin alone, exhibited a very weak interaction.

In these experiments, neither GST alone nor a GST fusion with the third FAP-1 PDZ domain showed detectable binding to ephrin B1. The identity of the 50 kD band recognized by GST-FAP-1 PDZ3 is not known but its apparent size does not correlate with any of the three known B ephrins. Consistent with this finding, the binding specificity of FAP-1 PDZ3, as previously determined using an oriented peptide library, is significantly different from that of FAP-1 PDZ5, with a preference towards target sequences such as the QSLV-COOH motif in the Fas antigen (1,33). The inability of the of FAP-1 PDZ3 domain to bind ephrin B1 indicates a degree of specificity in recognition of ephrin B1 by PDZ domains.

A hallmark of many PDZ domain binding sites is a requirement for a C-terminal hydrophobic residue that contacts the PDZ domain through its side chain and C-terminal carboxylate group (1, 38,39). The involvement of the C-terminal Val of ephrin B1 in specific binding to syntenin and FAP-1 PDZ5 was initially evaluated by expressing a deletion mutant of ephrin B1 lacking the terminal Val residue in Cos-1 cells. Removal of the C-terminal Val from full-length ephrin B1 abrogated its binding to both syntenin and FAP-1 PDZ5 GST fusion proteins (FIGS. 3B and 3C).

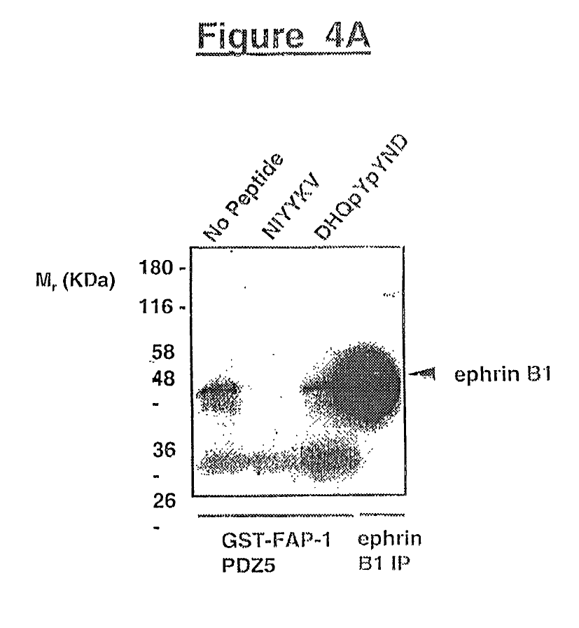

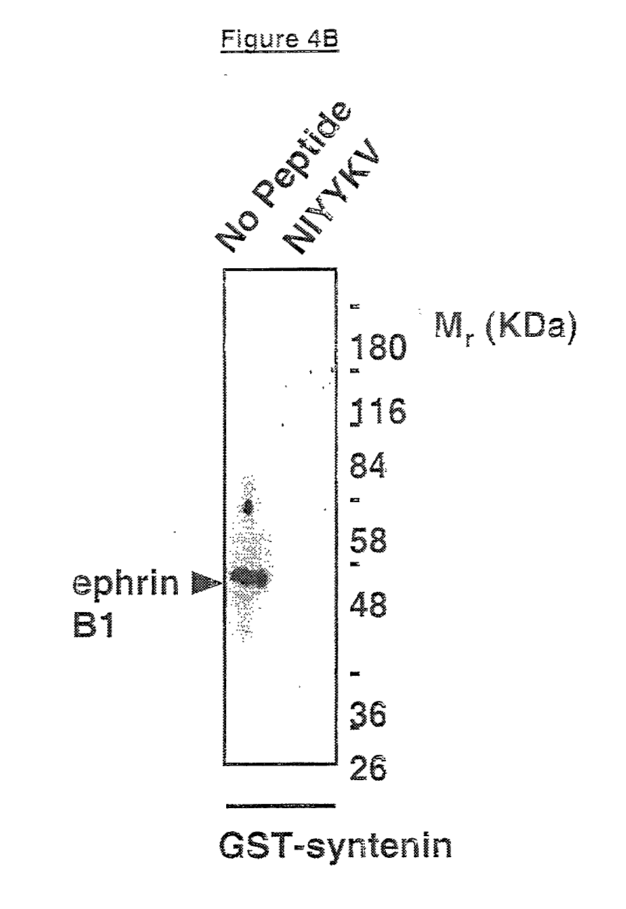

As an alternative approach towards investigating the specificity of ephrin B1 interactions with PDZ domain proteins, a specific peptide modeled on the C-terminus of B-type ephrins was employed in competition experiments. For this purpose, lysates of ephrin B1 -transfected cells were incubated with either GST-syntenin or GST-FAP-1 PDZ5 in the presence or absence of a peptide corresponding in sequence to the C-terminal six residues of B ephrins. The peptide successfully blocked syntenin and FAP-1 PDZ5 binding at a peptide concentration of 100 μM (FIGS. 4A and 4B). The addition of the unrelated peptide, DHQpYpYND (SEQ. ID. NO. 11), did not decrease binding, indicating the specificity of the peptide competition (FIG. 4A).

FAP-1 PDZ5 and Syntenin Display Differential Binding to Phosphopeptides

Binding of B ephrins to their cognate Eph B receptors, expression of an activated Src tyrosine kinase or treatment of ligand-expressing cells with PDGF results in tyrosine phosphorylation of residues in the ephrin cytoplasmic domain (26,27). Preliminary evidence based on specific substitutions of the Tyr residues in the ephrin B1 tail indicates that the two tyrosines at the -2 and -3 positions within the PDZ domain binding site are among the phosphorylation sites. To investigate whether tyrosine phosphorylation of these residues might affect PDZ domain binding, the C-terminal peptide used for the peptide competition described above was also synthesized such that either one or both of the -2 and -3 tyrosine residues were phosphorylated. The phosphorylated and unphosphorylated peptides were labeled with fluorescein and employed in fluorescence polarization experiments to obtain quantitative measurements of their affinities for FAP-1 and syntenin PDZ domains.

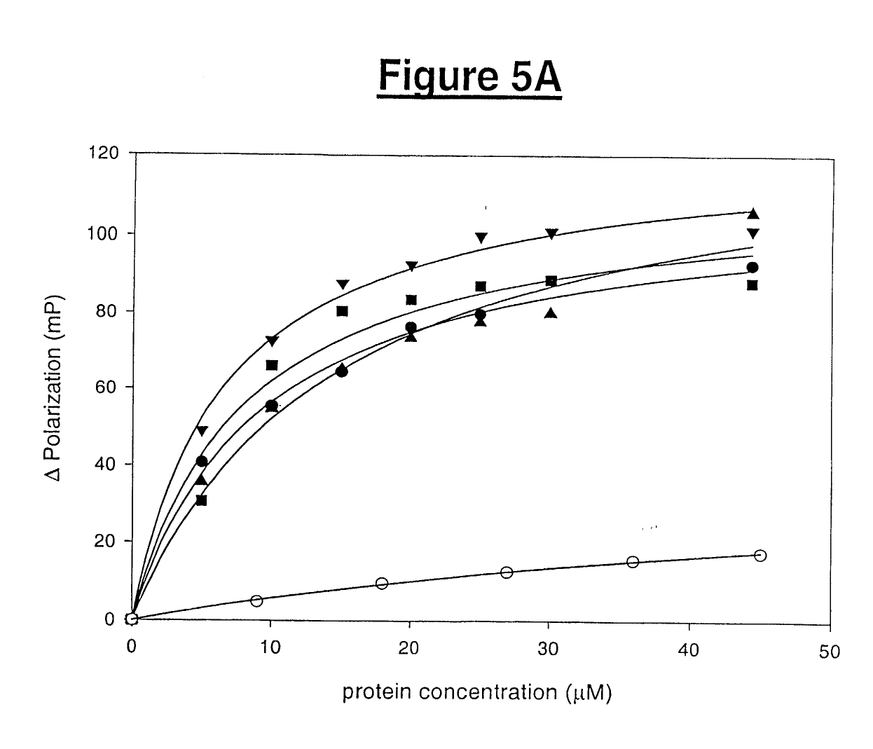

The GST-FAP-1 PDZ5 bound to a fluorescein-labeled NIYYKV (SEQ. ID. NO. 10) peptide with an affinity of 9.9+1.0 μM, while GST-FAP-1 PDZ3 binding was much weaker (65.0+9.6 μM) (FIG. 5A). This is consistent with the GST mixing experiments that indicated FAP-1 PDZ3 does not interact stably with ephrin B1. Similar results were obtained when binding to the three different phosphorylated peptides was investigated, indicating that alternative tyrosine phosphorylation states of the B ephrin C-terminal sequence had little effect on binding to GST-FAP-1 PDZ5. Similar binding affinity values of 6.8+0.8 μM, 15.4+3.4 μM and 8.4+2.5 μM were obtained for the NIpYYKV, NIYpYKV and NIpYpYKV (SEQ. ID. NO. 8, 9, and 7 respectively) peptides, respectively.

Fluorescence polarization experiments measuring GST-syntenin fusion protein binding to fluorescein-labeled NIYYKV (SEQ. ID. NO. 10) and NIpYYKV (SEQ. ID. NO. 8) peptides yielded nearly identical binding curves (FIG. 5B). Affinity values of 17.7 +1.2 μM and 15.4+0.5 μM were obtained, indicating that phosphorylation at the -3 position tyrosine does not significantly affect the PDZ-domain interaction. However, the GST-syntenin fusion protein bound the pYpYKV (SEQ. ID. NO. 12) peptide with a much lower affinity of 151.0+20.9 μM, indicating that phosphorylation at the -2 Tyr can have a detrimental effect on binding to syntenin. A similar low affinity interaction was observed for the YpYKV peptide.

Ephrin B1 and Syntenin Can Associate in Cells

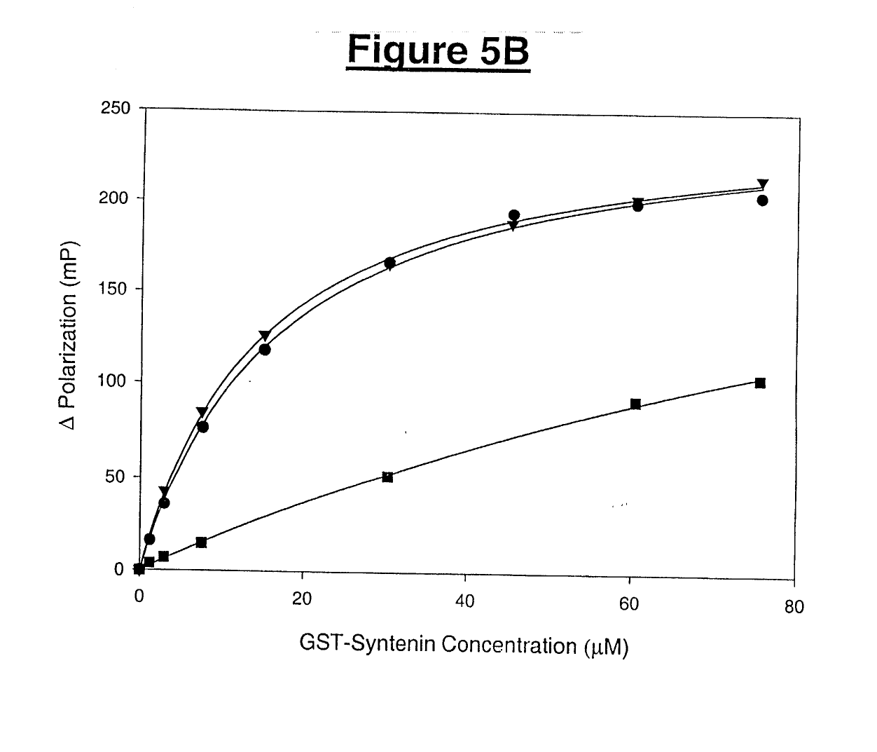

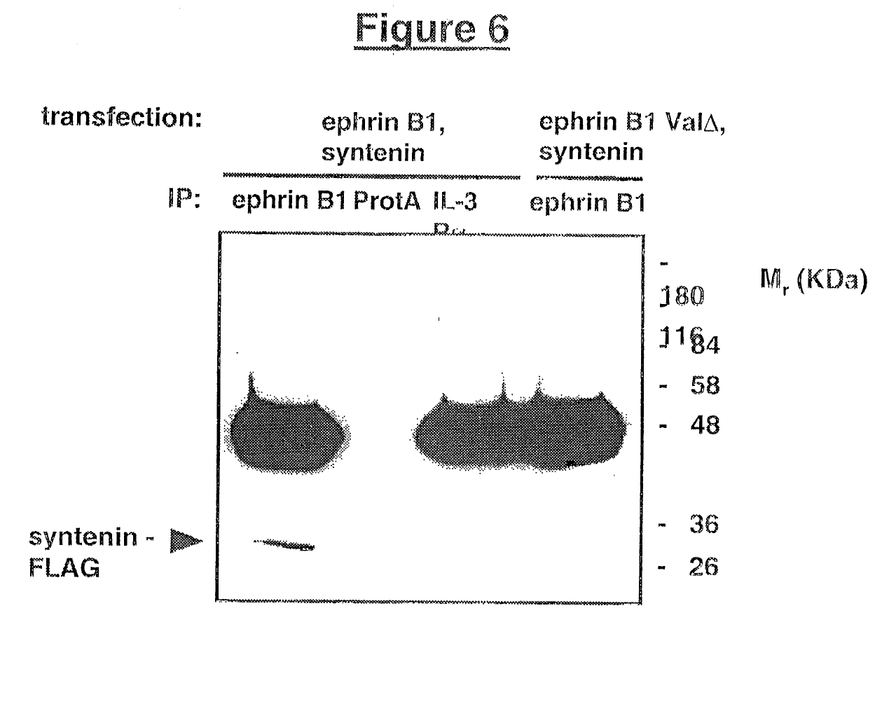

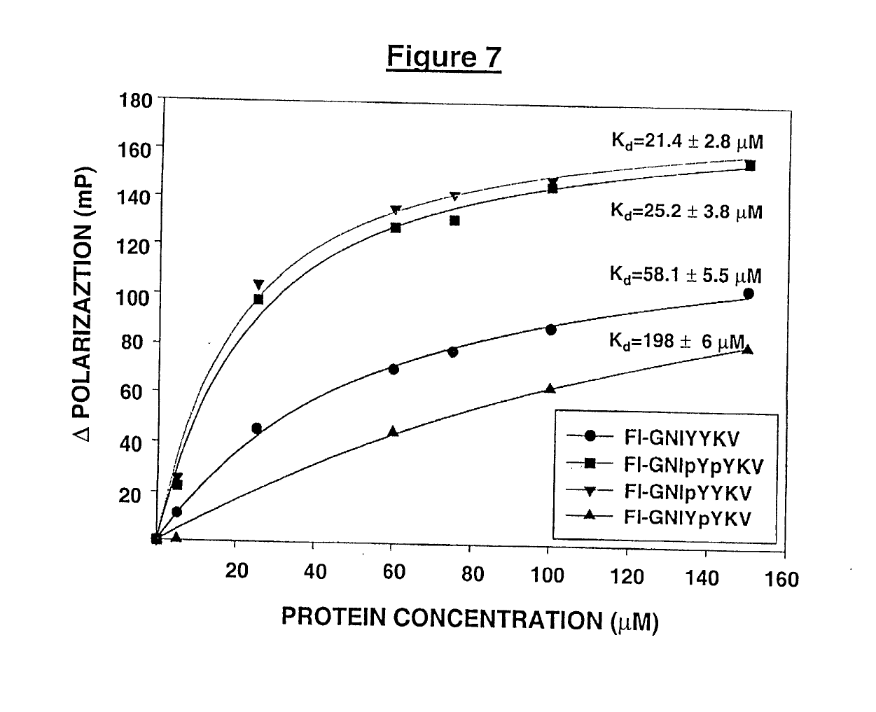

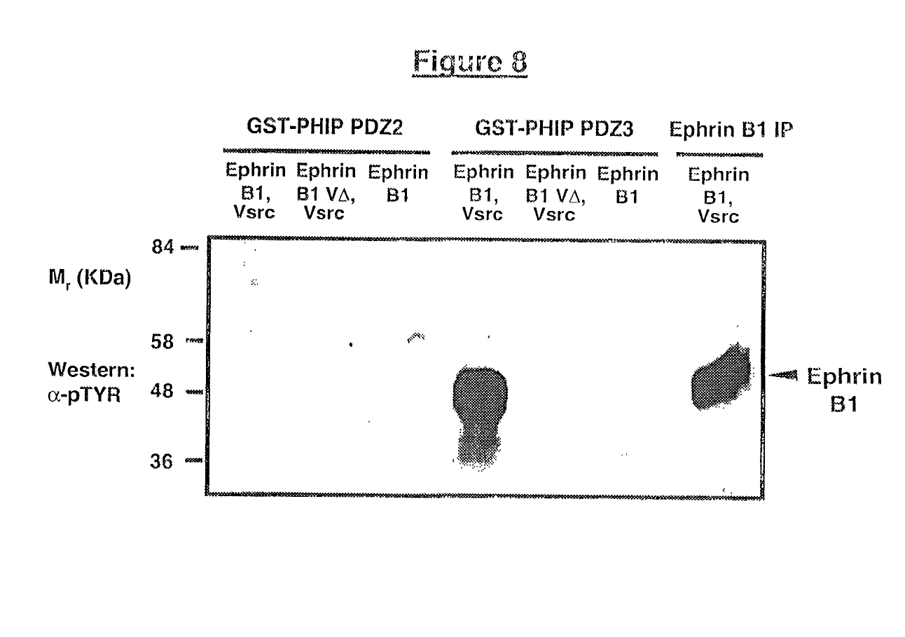

The possibility that B-type ephrins may interact with PDZ domain proteins in vivo was pursued by assaying whether ephrin B1 and syntenin associate when co-expressed in Cos-1 cells. In cells co-transfected with ephrin B1 and syntenin (tagged at its N-terminus with a FLAG epitope) immunoprecipitation of ephrin B1 specifically co-precipitated syntenin (FIG. 6). Precipitation with protein A sepharose alone or with an arbitrarily chosen antibody did not yield detectable syntenin, indicating that the interaction is specific. Further, co-immunoprecipitation experiments with the ephrin B1 Val deletion mutant, which fails to interact with PDZ domains in vitro, showed that ephrin B1 lacking the C-terminal Val did not detectably associate with syntenin (FIG. 6). While the truncated protein could be successfully immunoprecipitated by antibodies against ephrin B1, syntenin could not be co-immunoprecipitated with the mutant protein. These results demonstrate that ephrin B1 and syntenin can associate in cells, and show that an intact PDZ domain binding site in ephrin B1 is necessary for its interaction with syntenin in vivo.

DISCUSSION

In an effort to identify components of the cytoplasmic domain that may contribute to ephrin B function, it was demonstrated that the C-terminal residues of B ephrins constitute a binding site for PDZ domains, a class of protein module known to mediate specific protein-protein interactions. Several lines of evidence indicate that the C-terminal YYKV sequence, conserved among all 3 known B ephrins, represents a PDZ domain binding site. Firstly, a biotinylated peptide probe with a sequence corresponding to the C-terminal residues of ephrin B3 identified cDNAs coding for the known PDZ domain-containing proteins syntenin and GRIP, as well as a cDNA for PHIP, a novel PDZ domain-containing protein. In addition, a fourth PDZ-containing protein, FAP-1, was identified as a binding candidate based initially on the predicted binding specificity of its fifth PDZ domain.

Secondly, in vitro studies with syntenin and FAP-1 have demonstrated specific interactions of the PDZ domains of these proteins with the C-terminus of ephrin B 1. The finding that the C-terninal Val residue of ephrin B1 is absolutely required for these interactions indicates that binding occurs in a manner characteristic of other PDZ domain interactions with C-terminal target sequences. Similar results were also obtained from in vitro binding experiments with ephrin B2, suggesting that PDZ domain interactions may be common to all B ephrins. In vitro experiments were also performed with separate GST fusions of GRIP PDZ6 and GRIP PDZ7. Interactions with ephrin B1 or with the fluorescent GNIYYKV (SEQ. ID. NO. 13) peptide were not detected in GST-mixing and fluorescence polarization experiments. Binding to ephrin B1 may require both PDZ 6 and PDZ 7 of GRIP in a fashion reminiscent of the requirement of both syntenin PDZ domains for binding. Lastly, it was demonstrated that B ephrin-PDZ domain interactions can occur in vivo, since syntenin can be successfully co-immunoprecipitated with full-length ephrin B1 but not with ephrin B1 truncated in its PDZ domain target site.

The effect of the phosphorylation state of two adjacent tyrosines at positions -2 and -3 relative to the C-terminal Val of the PDZ domain target site was examined using a fluorescence polarization assay. Structural studies of PDZ domains have suggested that interactions between PDZ domains and residues at the -2 and -3 positions of the C-terminal target site confer binding specificity (38-40). In one case, modification of residues at these positions by serine phosphorylation has been reported to regulate PDZ domain binding. The specific association between the second PDZ domain of PSD-95 and the inward rectifier potassium (K+) channel Kir2.3 is disrupted by protein kinase A mediated phosphorylation of a key serine residue at the -2 position from the C-terminus of Kir2.3 (41). The results with B class ephrins and the PDZ domain proteins FAP-1 and syntenin suggest that the phosphorylation of residues within the PDZ domain binding site has different effects on different PDZ domains. The results with FAP-1 PDZ5 suggest that the PDZ domain residues which contact the tyrosines in the binding site of B ephrins are able to accommodate the addition of two phosphate groups. This is consistent with observations that the single PDZ domain of AF-6 binds an unphosphorylated peptide with the consensus target sequence AYYV (SEQ. ID. NO. 14) and a corresponding peptide phosphorylated at the -2 Tyr residue with approximately equal affinity. In contrast, GST-syntenin exhibited significantly decreased binding to peptides phosphorylated at the -2 residue of the PDZ domain binding site. These data indicate one mechanism through which tyrosine phosphorylation of ephrin B1 may regulate interactions with modular cytoplasmic proteins. Possible roles for PDZ domain-ephrin B associations can be proposed based on known fimctions of PDZ domains. Several examples have highlighted the importance of PDZ domain interactions in the proper localization and clustering of transmembrane proteins (42,43). For instance, the positioning of NMDA receptors and K⁺ channels at post-synaptic termini is likely dependent on specific interactions of these receptors with PDZ domain-containing proteins (34, 44-47). In Drosophila larvae, null mutations of the gene encoding the PDZ protein discs-large result in mislocalization of the Shaker K⁺ channel (48). Clustering of Shaker K⁺ channels via PDZ domain interactions has also been demonstrated in COS7 cells co-expressing the channel with either of its binding partners, PSD-95 or chapsyn 110 (49).

A requirement for correct localization and clustering figures prominently in the proposed functions of B class ephrins. Since ephrin B-EphB interactions involve direct cell-cell contact, ephrins must be present at sites of contact with receptor-expressing cells. This localization may be mediated by PDZ domain associations with the C-terminus of B ephrins. In this regard, it is of interest that PHIP is a close relative of PAR-3, a C. elegans protein that regulates asymmetry and polarity in the early embryo. It is possible that PHIP has a similar function in mammalian cells in controlling the asymmetric distribution of proteins with PDZ domain-binding motifs. Studies involving soluble forms of the extra-cellular domain of ephrins have revealed a requirement for ligand clustering in receptor activation. Whereas treatment of receptor-expressing cells with soluble versions of the ligands does not result in receptor activation and subsequent autophosphorylation, artificial aggregation of soluble ephrins by clustering antibodies allows activation of the receptor (18). Since co-culturing of ephrin-expressing cells with cells expressing Eph receptors leads to receptor activation, membrane-bound ligands must also become clustered in some manner. Furthermore, recent studies in a renal endothelial cell system have indicated that the state of ephrin B1 oligomerization is important in determining alternative receptor signaling complexes as well as attachment and assembly responses in the receptor-bearing cell (50). Although binding of both ligand dimers and higher order oligomers cause receptor autophosphorylation, only tetrameric forms of the ligand were able to induce the attachment response and stimulate the recruitment of low molecular weight phosphotyrosine phosphatase to the activated receptor. Given the known role of PDZ domains in the clustering of transmembrane proteins, PDZ domain interactions with ephrin B1 may play a role in the presentation of the ligand in the correct oligomeric form to elicit specific responses in the receptor-expressing cell.

Another role ascribed to PDZ domain-containing proteins is to act as a scaffold to organize signaling complexes. This is well illustrated by the function of the protein InaD in the photo-transduction pathway of the Drosophila compound eye. Key components of this cascade, including the transient receptor potential (TRP) calcium channel, the eye form of protein kinase C and phospholipase C-P are bound by the PDZ domains of InaD to form a compartmentalized signalling complex (51,52). Mutations in specific InaD PDZ domains that abolish binding result in defects in the kinetics of the phototransduction cascade. In the case of B ephrins, genetic evidence along with biochemical studies indicating that tyrosine residues in the intracellular domain become phosphorylated upon receptor binding or PDGF treatment has led to the hypothesis that the cytoplasmic tail of B ephrins may have an intrinsic signaling function (2,6,26,27). The phosphorylated tyrosine residues represent potential docking sites for proteins with phosphotyrosine recognition modules such as SH2 or PTB domains. Downstream components of this possible phosphotyrosine-dependent signaling pathway may be assembled around a PDZ domain-containing protein in a manner similar to the InaD complex. Furthermore, the PDZ domain-containing protein PSD-95 which associates with glutamate receptors and K⁺ channels also interacts through its PDZ domains with neuronal nitric oxide synthase and a Ras GTPase activating protein (p135 SynGAP) (53,54). PDZ domain-containing proteins may thereby serve as adaptors to directly activate signaling pathways. In this context, it is of interest that phosphorylation of the Tyr residues in the C-terminal ephrin B1 motif may regulate interactions with PDZ domains, as suggested by the results with syntenin.

Having illustrated and described the principles of the invention in a preferred embodiment, it should be appreciated to those skilled in the art that the invention can be modified in arrangement and detail without departure from such principles. All modifications coming within the scope of the following claims are claimed.

All publications, patents and patent applications referred to herein are incorporated by reference in their entirety to the same extent as if each individual publication, patent or patent application was specifically and individually indicated to be incorporated by reference in its entirety.

### FULL CITATIONS FOR REFERENCES REFERRED TO IN THE SPECIFICATION

1. Songyang, Z., Fanning, A. S., Fu, C., Xu, J., Marfatia, S. M., Chishti, A. H., Crompton, A., Chan, A. C., Anderson, J. M., and Cantley, L. C. (1997) Science 275, 73-77

2. George, S. E., Simokat, K., Hardin, J., and Chisolm, A. D. (1998) Cell 92, 633-643

3. Drescher, U. (1997) Current Biology 7, R799-R807

4. Eph Nomenclature Committee. (1997) Cell 90, 403-404

5. Henkemeyer, M., Marengere, L. E. M., McGlade, J., Olivier, J. P., Conlon, R. A., Holmyard, D. P., Letwin, K., and Pawson, T. (1994) Oncogene 9, 1001-1014

6. Henkemeyer, M., Orioli, D., Henderson, J. T., Saxton, T. M., Roder, J., Pawson, T., and Klein, R. (1996) Cell 86, 35-46

7. Drescher, U., Kremoser, C., Handwerker, C., Loschinger, J., Noda, M., and Bonhoeffer, F. (1995) Cell 82, 359-370

8. Nakamoto, M., Cheng, H. J., Friedman, G. C., Mclaughlin, T., Hansen, M. J., Yoon, C. H., O'Leary, D. D., and Flanagan, J. G. (1996) Cell 86, 755-766

9. Winslow, J. W., Moran, P., Valverde, J. Shih, A., Yuan, J. Q., Wong, S. C., Tsai, S. P., Goddard, A., Henzel, W. J., Hefti, F. et al. (1995) Neuron 14, 973-981

10. Cheng, H. J., Nakamoto, M., Bergemann, A. D., and Flanagan, J. G. (1995) Cell 82, 371-381.

11. Smith, A., Robinson, V., Patel, K., and Wilkinson, D. G. (1997) Current Biology 7, 561-570

12. Wang, H. U., and Anderson, D. J. (1996) Neuron 18, 383-396

13. Orioli, D., Henkemeyer, M., Lemke, G., Klein, R., and Pawson, T. (1996) EMBO J. 15, 6035-6049

14. Pandey, A., Shao, H., Marks, R. M., Polvemi, P. J., and Dixit, V. M. (1995) Science 268, 567-569

15. Wang, H. U., Chen, Z., and Anderson, D. J. (1998) Cell 93, 741-753

16. Cheng, H. J., and Flanagan, J. G. (1994) Cell 79, 157-168

17. Beckman, M. P., Cerretti, D. P., Baum, P., Vanden Bos, T., James, L., Farrah, T., Kozlosky, C., Hollingsworth, T., Shilling, H. Maraskovsky, E., et al. (1994) EMBO J. 13, 3757-3762

18. Davis, S., Gale, N. W., Aldrich, T. H., Maisonpierre, P. C., Lhotak, V., Pawson, T., Goldfarb, M., and Yancopoulos, G. D. (1994) Science 266, 816-819

19. Shao, H., Lou, L., Pandey, A., Pasquale, E. B., and Dixit, V. M. (1994) J. Biol. Chem. 269, 26606-26609

20. Bennett, B. D., Zeigler, F. C., Gu, Q. M., Fendly, B., Goddard, A. D., Gillet, N., and Matthews, W. (1995) Proc. Natl. Acad. Sci. USA 92, 1866-1870

21. Bergemann, A. D., Cheng, H. J., Brambilla, R., Klein, R., and Flanagan, J. G. (1995) Mol. Cell Biol. 15, 4921-4929

22. Gale, N. W., Flenniken, A., Compton, D. L., Jenkins, N., Copeland, N. G., Gilbert, D. J., Davis, S., Wilkinson, D. G., and Yancopoulos, G. D. (1996) Oncogene 13, 1343-1352

23. Gale, N. W., Holland, S. J., Valenzuela, D. M., Flenniken, A., Pan, L., Ryan, T. E., Henkemeyer, M., Strebhardt, K., Hirai, H., Wilkinson, D. G., Pawson, T., Davis, S., and Yancopoulos, G. D. (1996) Neuron 17, 9-19

24. Brambilla, R., Schnapp, A., Cassagranda, F., Labrador, J. P., Bergemann, A. D., Flanagan, J. g., Pasquale, E. B., and Klein, R. (1995) EMBO J 14, 3116-3126

25. Flenniken, A. M., Gale, N. W., Yancopolous, G. D., and Wilkinson, D. G. (1996) Dev. Biol. 179, 382-401

26. Holland, S. J., Gale, N. W., Mbamalu, G., Yancopoulos, G. D., Henkemeyer, M., and Pawson, T. (1996) Nature 383, 722-725

27. Bruckner, K., Pasquale, E. B. and Klein, R. (1997) Science 275, 1640-1643

28. Jones, T. L., Chong, L. D., Kim, J., Xu, R. H., Kung, H. F., and Daar, I. O. (1998) Proc. Natl. Acad. Sci. USA 95, 576-581

29. van der Geer, P., Wiley, S., Gish, G. D., and Pawson, T. (1996) Current Biology 6, 1435-1444

30. Sparks, A. B., Hoffman, N. G., McConnell, S. J., Fowlkes, D. M., and Kay, B. K. (1996) Nature Biotech. 14, 741-744

31. Saras, J., Claesson-Welsh, L., Heldin, C. H., and Gonez, L. J. (1994) J. Biol. Chem. 269, 24082-24089

32. Maekawa, K., Imagawa, N., Nagamatsu, M., and Harada, S. (1994) FEBS Lett. 337, 200-206

33. Sato, T., Irie, S., Kitada, S., and Reed, J. C. (1995) Science 268, 411-415

34. Dong, H., O'Brien, R. J., Fung, E. T., Lanahan, A. A., Worley, P.F., and Huganir, R. L. (1997) Nature 386, 279-284

35. Lin, J. J., Liang, H., and Fisher, P. B. (1996) Mol. and Cell. Diff. 4, 317-333

36. Grootjans, J. J., Zimmerman, P., Reekmans, G., Smets, A.,Degeest, G., Durr, J., and David, G. (1997) Proc. Natl. Acad. Sci. USA 94, 13683-13688

37. Etemad-Moghadam, B., Guo, S., and Kemphues, K. J. (1995) Cell 83, 743-752

38. Doyle, D. A., Lee, A., Lewis, J., Kim, E., Sheng, M., and Mackinnon, R. (1996) Cell 85, 1067-1076

39. Cabral, J. H., Petosa, C., Sutcliffe, M. J., Raza, S., Byron, O., Poy, F., Marfata, S. M., Chishti, A. H., and Liddington, R. C. (1996) Nature 382, 649-652

40. Daniels, D. L., Cohen, A. R., Anderson, J. M., and Brunger, A. T. (1998) Nature Struct. Biol. 5, 317-325

41. Cohen, N. A., Brenman, J. E., Snyder, S. H., and Bredt, D. S. (1996) Neuron 17, 759-767

42. Fanning, A. S., and Anderson, J. M. (1998) Protein Modules in Signal Transduction, Springer-Verlag, Berlin, pp. 209-233

43. Craven, S. E., and Bredt, D. S. (1998) Cell 93, 495-498

44. Kornau, H. C., Schenker, L. T., Kennedy, M. B., and Seeburg, P. H. (1995) Science 269, 1737-1740

45. Kim, E., Niethammer, M., Rothschild, A., Jan, Y. N., and Sheng, M. (1995) Nature 378, 85-88.

46. Irie, M., Hata, Y., Takeuchi, K., Ichtchenko, A., Toyoda, Hirao, K., Takai, Y., Rosahl, T. W., and Sudhof, T. C. (1997) Science 277, 1511-1515

47. Sprengel, R., Suchanek, B., Amico, C., Brusa, R., Burnasher, N., Rozov, A., Hvalby, O., Jensen, V., Paulson, O., Anderson, P. et al. (1998) Cell 92, 279-289

48. Tejedor, F. J., Bokhari, A., Rogero, O., Gorczyca, M., Zhang, J., Kim, E., Sheng, M., and Bodnik, V. (1997) J. Neuro. Sci. 17, 152-159

49. Kim, E., Cho, K., Rothschild, A., and Sheng, M. (1996) Neuron 17, 103-113

50. Stein, E., Lane, A. A., Cerreti, P., Schoecklmann, H. O., Schroff, A. D., Van Etten, V. L., and Daniel, T. O. (1998) Genes Dev. 12, 667-678

51. Chevesich, J., Kreuz, A. J., and Montell, C. (1997) Neuron 18, 95-105

52. Tsunoda, S., Sierralta, J., Sun, Y., Bodner, R., Suzuki, E., Becker, A., Socolich, M., and Zuker, C. S. (1997) Nature 388, 243-249

53. Brenman, J. E., Chao, D. S., Gee, S. H., McGee, A. W., Craven, S. E., Santillano, D. R., Huang, F., Xia, H., Peters, M. F., Froehner, S. C., and Bredt, D. S. (1996) Cell 84, 757-767

54. Chen, H. J., Rojas-Soto, M., Oguni, A., and Kennedy, M. B. (1998) Neuron 20, 895-904

55. Higgins, D., Thompson, J., and Gibson, T. (1994) Nucleic Acids Res. 22, 4673-4680

We claim:

1. An isolated complex comprising a B class ephrin and a PDZ domain containing protein.

2. An isolated complex as claimed in claim 1 wherein the B class ephrin is ephrin B1 or ephrin B3.

3. An isolated complex as claimed in claim 1 or 2 wherein the PDZ domain containing protein is GRIP, GRIP PDZ6 and PDZ 7 of SEQ.ID.NO. 22 and 23, FAP-1 PDZ of SEQ. ID. NO. 21, amino acids residues 1 to 299 of syntenin, syntenin PDZ1 and PDZ2 of SEQ. ID. NO. 26 and 27, PHIP PDZ2 of SEQ. ID. NO. 24; and PHIP PDZ3 of SEQ. ID. NO. 25.

4. An isolated complex as claimed in claim 3 which is ephrin B3/GRIP; ephrin B3/GRIP PDZ6 and PDZ 7 of SEQ.ID.NO. 22 and 23; ephrin B1/FAP-1 PDZ of SEQ. ID. NO. 21; ephrin B1 or B3/syntenin PDZ1 and PDZ2 of SEQ. ID. NO. 26 and 27; ephrin B1 or B3/ residues 1-299 of syntenin; ephrin B1 or B3/PHIP PDZ2 of SEQ. ID. NO. 24, ephrin B1 or B3/PHIP PDZ3 of SEQ. ID. NO. 25.

5. A peptide derived from the PDZ binding domain of a B class ephrin.

6. A synthetic peptide of the formula I which interferes with the interaction of a B class ephrin and a PDZ domain containing protein:
X-X¹-X² -K-V I
wherein X represents 0 to 70 amino acids, and each of X¹ and X² represent tyrosine or phosphotyrosine.

7. A peptide as claimed in claim 6 wherein X represents 2 to 20 amino acids.

8. A peptide as claimed in claim 7 wherein X represents NI, GNI, CPHYEKVSGDYGHPVYIVQ(E,D)(M,G)PPQSP(A,P)A (SEQ.ID. NO. 2), GDYGHPVYIVQ(E,D)(M,G)PPQSP(A,P)A (SEQ.ID. NO. 3), PPQSP(A,P)A (SEQ.ID. NO. 4), GPPQSPPNI (SEQ.ID. NO.).

9. A peptide as claimed in claim 7 which is YYKV (SEQ ID. NO. 5), GPPQSPPNIpYYKV (SEQ ID. NO. 6), NIpYpYKV (SEQ ID. NO. 7), NIpYYKV (SEQ ID. NO. 8), NIYpYKV (SEQ ID. NO. 9), NIYYKV (SEQ ID. NO. 10), GNIYYKV (SEQ ID. NO. 28), GNIpYpYKV (SEQ ID. NO. 29), GNIpYYKV (SEQ ID. NO. 30), or GNIYpYKV (SEQ ID. NO. 31).

10. A complex comprising a peptide as claimed in claim 6, 7, 8, or 9 and a PDZ domain containing protein.

11. A complex as claimed in claim 10 wherein the PDZ domain containing protein is GRIP, GRIP PDZ6 and PDZ 7 of SEQ.ID.NO. 22 and 23, FAP-1 PDZ of SEQ. ID. NO. 21, amino acids residues 1 to 299 of syntenin, syntenin PDZ1 and PDZ2 of SEQ. ID. NO. 26 and 27, PHIP PDZ2 of SEQ. ID. NO. 24; and PHIP PDZ3 of SEQ. ID. NO. 25.

12. A complex as claimed in claim 10 which is FAP-1 PDZ of SEQ. ID. NO. 21/NIpYYKV, FAP-1 PDZ of SEQ. ID. NO. 21/NIpYpYKV, syntenin/NIYYKV, syntenin/NIpYYKV, syntenin PDZ1 and PDZ2 of SEQ. ID. NO. 26 and 27/NIYYKV, syntenin PDZ1 and PDZ2 of SEQ. ID. NO. 26 and 27/ NIpYYKV, PHIP PDZ3 of SEQ. ID. NO. 25/GNIpYpYKV, or PHIP PDZ3 of SEQ. ID. NO. 25/GNIpYYKV.

13. A method of modulating the interaction of a B class ephrin and a PDZ domain containing protein comprising administering an effective amount of a complex as claimed in claim 1.

14. A method of modulating the interaction of a B class ephrin and a PDZ domain containing protein comprising administering an effective amount of a peptide as claimed in claim 6.

15. A method for identifying a substance that binds to a complex as claimed in claim 1 comprising: (a) reacting the complex with at least one substance which potentially can bind with the complex, under conditions which permit binding of the substance and complex; and (b) detecting binding, wherein detection of binding indicates the substance binds to the complex.

16. A method as claimed in claim 15 wherein binding is detected by assaying for substance-complex conjugates, or for activation of the B class ephrin B or PDZ domain containing protein.

17. A method for evaluating a compound for its ability to modulate the interaction of a B class ephrin and a PDZ domain containing protein which comprises providing a complex as claimed in claim 1, 2 or 3, with a substance which binds to the complex, and a test compound under conditions which permit the formation of conjugates between the substance and complex, and removing and/or detecting conjugates.

18. A method for evaluating a compound for its ability to modulate the interaction of a B class ephrin and a PDZ domain containing protein which comprises (a) providing a B class ephrin and a PDZ domain containing protein, and a test compound, under conditions which permit binding of the B class ephrin and PDZ domain containing protein; and (b) detecting binding, wherein the detection of increased or decreased binding relative to binding in the absence of the test compound indicates that the test compound modulates the interaction of a B class ephrin and a PDZ domain containing protein.

19. A method of modulating the interaction of a B class ephrin and a PDZ domain containing protein comprising changing the terminal amino acid Val in a B class ephrin.

20. Use of a complex as claimed in claim 1 or a peptide as claimed in claim 6 in the preparation of a medicament to modulate the interaction of a B class ephrin and a PDZ domain containing protein.

21. Use of a complex as claimed in claim 1 or a peptide as claimed in claim 6 in the preparation of a medicament to modulate cellular processes of cells associated with B class ephrins or PDZ domain containing proteins.

22. A use as claimed in claim 21 wherein the cellular processes are axonogenesis, nerve cell interactions, and regeneration of nerve cells.

23. A composition comprising a complex as claimed in claim 1 or a peptide as claimed in claim 6, and a pharmaceutically acceptable carrier, excipient or diluent effective for administration to individuals suffering from disorders associated with a B class ephrin.

24. A method for modulating proliferation, growth, or differentiation of cells associated with B class ephrins or PDZ domain containing proteins comprising introducing into the cells a complex as claimed in claim 1 or a peptide as claimed in claim 6.

25. A method for treating proliferative or differentiative disorders associated with B class ephrins or PDZ domain containing proteins using a composition as claimed in claim 23.

26. An isolated protein comprising the amino acid sequence of SEQ. ID. NO.1.

27. A truncation, an analog, an allelic or species variation of a protein as claimed in claim 26, or a protein having substantial sequence identity with the protein as claimed in claim 26.

28. A fusion protein comprising an isolated protein as claimed in claim 26 conjugated to a protein.

29. Antibodies having specificity against an epitope of a protein as claimed in claim 26.

30. A method for identifying a substance which binds to a protein as claimed in claim 26 comprising reacting the protein with at least one substance which potentially can bind with the protein, under conditions which permit the binding of the substance and protein, and detecting binding, wherein the detection of binding indicates that the substance binds to the protein.

31. A method for evaluating a compound for its ability to modulate the biological activity of a protein as claimed in claim 26 comprising providing the protein, a substance which binds to the protein, and a test compound under conditions which permit binding of the substance and protein, and detecting binding, wherein the detection of increased or decreased binding relative to binding detected in the absence of the test compound indicates that the test compound modulates the activity of the protein.

32. A method of conducting a drug discovery business comprising:
(a) providing one or more assay systems for identifying agents by their ability to inhibit or potentiate the interaction of an ephrin and a PZD domain of an intracellular protein;
(b) conducting therapeutic profiling of agents identified in step (a), or further analogs thereof, for efficacy and toxicity in animals; and
(c) formulating a pharmaceutical preparation including one or more agents identified in step (b) as having an acceptable therapeutic profile.

33. The method of claim 32, including a step of establishing a distribution system for distributing the pharmaceutical preparation for sale.

34. The method of claim 32, including establishing a sales group for marketing the pharmaceutical preparation.

35. A method of conducting a target discovery business comprising:
(a) providing one or more assay systems for identifying agents by their ability to inhibit or potentiate the interaction of an ephrin and a PZD domain of an intracellular protein;
(b) (optionally) conducting therapeutic profiling of agents identified in step (a) for efficacy and toxicity in animals; and
(c) licensing, to a third party, the rights for further drug development and/or sales for gents identified in step (a), or analogs thereof.
